(19) United States
(12) Patent Application Publication
(10) Pub. No.: US 2006/0281798 A1
(43) Pub. Date: Dec. 14, 2006

(54) Methods of using thiazolidinedithione derivatives

(75) Inventors: Zaihui Zhang, Richmond (CA); Timothy Daynard, Vancouver (CA); Gabriel Bela Kalmar, Rindmond (CA); Jun Yan, Coquitlam (CA); David L. Charest, Vancouver (CA)

Correspondence Address: SEED INTELLECTUAL PROPERTY LAW GROUP PLLC, 701 FIFTH AVE, SUITE 6300, SEATTLE, WA, 98104, US

(73) Assignee: QLT Inc., Vancouver (CA)

(21) Appl. No.: 10/515,087

(22) PCT Filed: May 16, 2003
(86) PCT No.: PCT/CA03/00741

§ 371 Date: Aug. 30, 2005

(30) Foreign Application Priority Data
May 17, 2002 (US) 60381638

(51) Int. Cl. A61K 31/427; A61K 31/426; C07D 417/02; C07D 277/26

(52) U.S. Cl. 514/369; 548/183

(57) ABSTRACT

Methods of using thiazolidinedithione derivatives to treat cancer, neurodegenerative disease, diabetes, renal disease or inflammation in a mammal and pharmaceutical compositions containing such derivatives are disclosed.

### FIELD OF THE INVENTION

This invention is directed to methods of using thiazolidinedithione derivatives.

### BACKGROUND OF THE INVENTION

Protein phosphorylation is a common regulatory mechanism used by cells to selectively modify proteins carrying regulatory signals from outside the cell to the nucleus. The proteins that execute these biochemical modifications are a group of enzymes known as protein kinases and protein phosphatases. They may further be defined by the substrate residue that they target for phosphorylation. Kinases and protein kinase pathways are involved in most cell signaling, and many of the pathways play a role in human disease. Protein tyrosine phosphorylation is an important mechanism for transmitting extracellular stimuli in biochemical and cellular events such as cell attachment, mitogenesis, differentiation and migration (see e.g., Li et al., Seminars in Immunology (2000), Vol.12, pp. 7-584, and Neel et al., Current Opinion in Cell Biology (1997), Vol. 9, pp.193-204).

Phosphorylation is important in signal transduction mediated by receptors via extracellular biological signals such as growth factors or hormones. For example, many oncogenes are kinases or phosphatases, i.e. enzymes that catalyze protein phosphorylation or dephosphorylation reactions or are specifically regulated by phosphorylation. In addition, a kinase or phosphatase can have its activity regulated by one or more distinct kinase or phosphatases, resulting in specific signaling cascades.

All protein tyrosine phosphatases (PTPs) have a conserved catalytic domain characterized by a signature sequence (I/V)HCXXGXX(S/T). Biochemical and kinetic studies have demonstrated that the cysteine residue found in this signature sequence is essential for catalytic activity of PTPs since this mutation of this cysteine completely abolishes PTP activity. See, Flint, A. J., et al., Proceedings of the National Academy of Sciences of the United States of America 94 (1997), pp.1680-1685.

All protein tyrosine kinases (PTKs) have multiple conserved regions within the catalytic region (Hanks, S.K. et al, “Protein kinases 6. The eukaryotic protein kinase superfamily: kinase (catalytic) domain structure and classification”, FASEB J. (1995), Vol. 9, No. 8, pp. 576-96) as well as variable regions relating to the specific role of the kinase. For this reason, compounds that inhibit kinases may be selective for a single kinase, or a single group of kinases.

### DESCRIPTION OF THE RELATED ART

PCT Published Patent Application, WO 99/61467 (McGill University), describes agents that interfere with the binding of PTPN12 (PTP-PEST) to domains of signalling proteins as inhibitors of cell migration and/or of focal adhesion.

PCT Published Patent Application, WO 00/36111 (McGill University) describes methods of utilizing PTPN2 (TC-PTP) for screening.

U.S. Pat. No. 6,262,044 (Novo Nordisk) describes certain protein tyrosine phosphatase inhibitors and provides a detailed description of the discovery of protein tyrosine phosphatases and their pathophysiological roles. U.S. Pat. No. 5,726,027 by Olefsky, Jerald M. describes a screening method for identifying compounds which affect the binding protein tyrosine phosphatase IB (PTPN1) to phosphorylate insulin receptor.

Gorishnii, V. Ya. et al., Farm. Zh. (Kiev) (2001), Vol. 2, pp. 64-67, and Gorishnyi, V. Ya. et al., Farm. Zh. (Kiev) (1995), Vol. 4, pp. 50-53, discloses 4-oxo-2-thioxothiazolidine derivatives useful in treating inflammation. PCT Published Patent Application WO 00/76988 (Wamer-Lambert) discloses 4-oxo-2-thioxothiazolidine derivatives useful as amyloid aggregation inhibitors and in imaging amyloid deposits. European Patent Specification 0 047 109 (Ono Pharmaceuticals) discloses 4-oxo-2-thioxothiazolidine derivatives useful in inhibiting aldose reductase.

### SUMMARY OF THE INVENTION

This invention is directed to the use of certain thiazolidinedithione derivatives in treating hyperproliferative disorders, e.g. cancer, inflammation, etc. in a mammal. Of particular interest are hyperproliferative disorders associated with cellular modulation of protein phosphorylation states, i.e. altered activity of phosphorylation modifying enzyme(s), e.g. protein tyrosine kinases and protein tyrosine phosphatases.

In embodiment, compounds and pharmaceutical compositions of the invention are used to inhibit the activity of PTPN12, PTPN2, PRKD2, PTPN1 and/or GSK3β. These enzymes have been associated with alterations in the phosphorylation state of cellular proteins.

Accordingly, one aspect of this invention provides a method of treating hyperproliferative disorders in a mammal, which method comprises administering to the mammal in need thereof a therapeutically effective amount of a compound of formula (I):

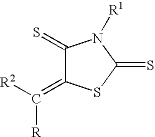

wherein:

R is heterocyclyl;

R¹ is hydrogen, alkyl, alkenyl, aryl, aralkyl, aralkenyl, cycloalkyl, cydoalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;

R² is hydrogen, alkyl, aralkyl, aryl, haloalkyl, cycloalkyl, cycloalkylalkyl, heterocyclyl or heterocyclylalkyl;

each R⁵ is independently an optionally substituted straight or branched alkylene or alkenylene chain;

each R⁶ is independently hydrogen, alkyl, alkenyl, cycloalkyl, cycloalkylalkyl, aralkyl or aryl; and

each R⁷ is independently hydrogen, alkyl or aralkyl;

as a single stereoisomer, a mixture of stereoisomers, or as a racemic mixture of stereoisomers; or as a solvate or polymorph; or as a pharmaceutically acceptable salt thereof.

In another aspect, this invention provides a method of treating a mammal having a disorder or condition associated with hyperproliferation and tissue remodelling or repair, wherein said method comprises administering to the mammal having the disorder or condition a therapeutically effective amount of a compound of formula (I):

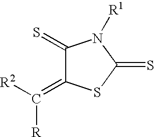

wherein:

R is heterocyclyl;

R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;

R² is hydrogen, alkyl, aralkyl, aryl, haloalkyl, cycloalkyl, cycloalkylalkyl, heterocyclyl or heterocyclylalkyl;

each R⁵ is independently an optionally substituted straight or branched alkylene or alkenylene chain;

each R⁶ is independently hydrogen, alkyl, alkenyl, cycloalkyl, cycloalkylalkyl, aralkyl or aryl; and

each R⁷ is independently hydrogen, alkyl or aralkyl;

as a single stereoisomer, a mixture of stereoisomers, or as a racemic mixture of stereoisomers; or as a solvate or polymorph; or as a pharmaceutically acceptable salt thereof.

In another aspect, this invention provides a method of treating a mammalian cell with a compound of formula (I):

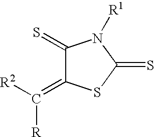

wherein:

R is heterocyclyl;

R¹ is hydrogen, alkyl, alkenyl, aryl, aralkyl, aralkenyl, cycloalkyl, cycloalkylalkyl, haloalkyl, haloalkenyl, heterocydylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;

R² is hydrogen, alkyl, aralkyl, aryl, haloalkyl, cycloalkyl, cycloalkylalkyl, heterocyclyl or heterocydylalkyl;

each R⁵ is independently an optionally substituted straight or branched alkylene or alkenylene chain;

each R⁶ is independently hydrogen, alkyl, alkenyl, cycloalkyl, cycloalkylalkyl, aralkyl or aryl; and

each R⁷ is independently hydrogen, alkyl or aralkyl;

as a single stereoisomer, a mixture of stereoisomers, or as a racemic mixture of stereoisomers; or as a solvate or polymorph; or as a pharmaceutically acceptable salt thereof;

wherein the method comprises administering the compound of formula (I) to a mammalian cell and the compound of formula (I) is capable of inhibiting the activity of PTPN12, PTPN2, PTPN1, PRKD2, and/or GSK3β, within the mammalian cell.

In another aspect, this invention provides a pharmaceutical composition useful in treating cancer or inflammation in a human, wherein the pharmaceutical composition comprises a pharmaceutically acceptable carrier, diluent or excipient and a compound of formula (I):

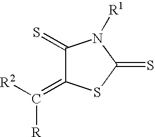

wherein:

R is heterocyclyl;

R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;

R² is hydrogen, alkyl, aralkyl, aryl, haloalkyl, cycloalkyl, cycloalkylalkyl, heterocyclyl or heterocyclylalkyl;

each R⁵ is independently an optionally substituted straight or branched alkylene or alkenylene chain;

each R⁶ is independently hydrogen, alkyl, alkenyl, cycloalkyl, cycloalkylalkyl, aralkyl or aryl; and

each R⁷ is independently hydrogen, alkyl or aralkyl;

as a single stereoisomer, a mixture of stereoisomers, or as a racemic mixture of stereoisomers; or as a solvate or polymorph; or as a pharmaceutically acceptable salt thereof;

provided, however, that when R¹ and R² are both hydrogen, R can not be unsubstituted thien-2-yl.

In another aspect, this invention provides compounds of formula (I):

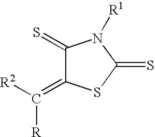

wherein:

R is heterocyclyl;

R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;

R² is hydrogen, alkyl, aralkyl, aryl, haloalkyl, cycloalkyl, cycloalkylalkyl, heterocyclyl or heterocyclylalkyl;

each R⁵ is independently an optionally substituted straight or branched alkylene or alkenylene chain;

each R⁶ is independently hydrogen, alkyl, alkenyl, cycloalkyl, cycloalkylalkyl, aralkyl or aryl; and

each R⁷ is independently hydrogen, alkyl or aralkyl;

as a single stereoisomer, a mixture of stereoisomers, or as a racemic mixture of stereoisomers; or as a solvate or polymorph; or as a pharmaceutically acceptable salt thereof;

provided, however, that when R¹ and R² are both hydrogen, R can not be unsubstituted thien-2-yl; and

provided, however, that when R¹ and R² are both hydrogen; R can not be unsubstituted furan-2-yl; 3-nitrofuran-2-yl, 4-nitrofuran-2-yl or 4-bromofuran-2-yl.

In another aspect of the invention, the use of compounds and/or pharmaceutical compositions of the invention for the treatment of cancer or inflammation in provided.

In another aspect of the invention, compounds and/or pharmaceutical compositions are provided for use in treating colon or colorectal cancer.

In another aspect of the invention, the use of compounds and/or pharmaceutical compositions of the invention in the manufacture of medicaments for the treatment of disorders associated with PTPN12, PTPN2, PTPN1, PRKD2, or GSK3β expression.

In another aspect of the invention, the use of compounds and/or pharmaceutical compositions are provided for the treatment of disorders associated with hyperproliferation, tissue remodelling, and/or tissue repair.

In another aspect of the invention, the use of compounds and/or pharmaceutical compositions of the invention for the treatment of diabetes, or in the manufacture of medicaments for the treatment of diabetes, are provided.

In another aspect of the invention, the use of compounds and/or pharmaceutical compositions are provided for the treatment of disorders associated with PTPN12, PTPN2, PRKD2, or GSK3β expression.

### DETAILED DESCRIPTION OF THE INVENTION

Definitions

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art. As used in the specification and appended claims, unless specified to the contrary, the following terms have the meaning indicated.

“Alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by hydroxy, alkoxy, aryloxy, haloalkoxy, cyano, nitro, mercapto, alkylthio, —N═N—O—R⁷, —N(R⁶)₂, —C(O)OR⁶, —C(O)N(R⁶)₂, —N(R⁶)C(O)R⁶, —C(O)R⁶, —N(R⁶)C(O)N(R⁶)₂, —OC(O)N(R⁶)₂, —N(R⁶)C(O)OR⁶, —S(O)ₜR⁶ (where t is 0 to 2), —S(O)ₜN(R⁶)₂ (where t is 0 to 2), or —N(R⁶)S(O)ₜR⁶ (where t is 0 to 2) where each R⁶ is independently hydrogen, alkyl, alkenyl, cycloalkyl, cycloalkylalkyl, aralkyl or aryl, and R⁷ is hydrogen, alkyl or aralkyl. Unless stated otherwise specifically in the specification, it is understood that for radicals, as defined below, that contain a substituted alkyl group that the substitution can occur on any carbon of the alkyl group.

“Alkenyl” refers to a straight or branched hydrocarbon chain radical consisting solely of carbon and hydrogen atoms, containing at least one double bond, having from two to eight carbon atoms, and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by hydroxy, alkoxy, haloalkoxy, cyano, nitro, mercapto, alkylthio, cycloalkyl, —N(R⁶)₂, —C(O)OR⁶, —C(O)N(R⁶)₂, —N(R⁶)—C(O)—R⁶, —C(O)R⁶, —N(R⁶)C(O)N(R⁶)₂, —OC(O)N(R⁶)₂, —N(R⁶)C(O)OR⁶, —S(O)ₜR⁶ (where t is 0 to 2), —S(O)ₜN(R⁶)₂ (where t is 0 to 2), or —N(R⁶)S(O)ₜR⁶ (where t is 0 to 2) where each R⁶ is independently hydrogen, alkyl, alkenyl, cycloalkyl, cycloalkylalkyl, aralkyl or aryl. Unless stated otherwise specifically in the specification, it is understood that for radicals, as defined below, that contain a substituted alkenyl group that the substitution can occur on any carbon of the alkenyl group.

“Aryl” refers to a phenyl or naphthyl radical. Unless stated otherwise specifically in the specification, the term “aryl” or the prefix “ar-” (such as in “aralkyl”) is meant to include aryl radicals optionally substituted by one or more substituents selected from the group consisting of hydroxy, alkoxy, aryloxy, haloalkoxy, cyano, nitro, mercapto, alkylthio, cycloalkyl, —N(R⁶)₂, —C(O)OR⁶, —C(O)N(R⁶)₂, —N(R⁶)C(O)R⁶, —C(O)R⁶, —N(R⁶)C(O)N(R⁶)₂, —OC(O)N(R⁶)₂, —N(R⁶)C(O)OR⁶, —S(O)ₜR⁶ (where t is 0 to 2), —S(O)ₜN(R⁶)₂ (where t is 0 to 2), or —N(R⁶)S(O)ₜR⁶ (where t is 0 to 2) where each R⁶ is independently hydrogen, alkyl, alkenyl, cycloalkyl, cycloalkylalkyl, aralkyl or aryl.

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkenyl” refers to a radical of the formula —R_{c}R_{b} where R_{c} is an alkenyl radical as defined above and R_{b} is one or more aryl radicals as defined above, e.g., 3-phenylprop-1-enyl, and the like. The aryl radical(s) and the alkenyl radical may be optionally substituted as described above.

“Alkylene” and “alkylene chain” refer to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of aryl, halo, hydroxy, alkoxy, haloalkoxy, cyano, nitro, mercapto, alkylthio, cycloalkyl, —N(R⁶)₂, —C(O)OR⁶, —C(O)N(R⁶)₂, —N(R⁶)C(O)R⁶, —C(O)R⁶, —N(R⁶)C(O)N(R⁶)₂, —OC(O)N(R⁶)₂, —N(R⁶)C(O)OR⁶, —S(O)ₜR⁶ (where t is 0 to 2), —S(O)ₜN(R⁶)₂ (where t is 0 to 2), or —N(R⁶)S(O)ₜR⁶ (where t is 0 to 2) where each R⁶ is independently hydrogen, alkyl, alkenyl, cycloalkyl, cycloalkylalkyl, aralkyl or aryl. The alkylene chain may be attached to the rest of the molecule through any two carbons within the chain.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of aryl, halo, hydroxy, alkoxy, haloalkoxy, cyano, nitro, mercapto, alkylthio, cycloalkyl, —N(R⁶)₂, —C(O)OR⁶, —C(O)N(R⁶)₂, —N(R⁶)C(O)R⁶, —C(O)R⁶, —N(R⁶)C(O)N(R⁶)₂, —OC(O)N(R⁶)₂, —N(R⁶)C(O)OR⁶, —S(O)ₜR⁶ (where t is 0 to 2), —S(O)ₜN(R⁶)₂ (where t is 0 to 2), or —N(R⁶)S(O)ₜR⁶ (where t is 0 to 2) where each R⁶ is independently hydrogen, alkyl, alkenyl, cycloalkyl, cycloalkylalkyl, aralkyl or aryl. The alkenylene chain may be attached to the rest of the molecule through any two carbons within the chain.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cydopentyl, cyclohexyl, decalinyl and the like. Unless otherwise stated specifically in the specification, the tern “cycloalkyl” is meant to include cycloalkyl radicals which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R⁶)₂, —C(O)OR⁶, —C(O)N(R⁶)₂, —N(R⁶)C(O)R⁶, —C(O)R⁶, —N(R⁶)C(O)N(R⁶)₂, —OC(O)N(R⁶)₂, —N(R⁶)C(O)OR⁶, —S(O)ₜR⁶ (where t is 0 to 2), —S(O)ₜN(R⁶)₂ (where t is 0 to 2), or —N(R⁶)S(O)ₜR⁶ (where t is 0 to 2) where each R⁶ is independently hydrogen, alkyl, alkenyl, cycloalkyl, cycloalkylalkyl, aralkyl or aryl.

“Cycloalkylalkyl” refers to a radical of the formula —RₐR_{d} where Rₐ is an alkyl radical as defined above and R_{d} is a cycloalkyl radical as defined above. The alkyl radical and the cycloalkyl radical may be optionally substituted as defined above.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Heterocyclyl” refers to a stable 3- to 15-membered ring radical that consists of carbon atoms and from one to five heteroatoms selected from the group consisting of nitrogen, oxygen and sulfur. For purposes of this invention, the heterocyclyl radical may be a monocyclic, bicyclic or tricyclic ring system, which may include fused or bridged ring systems; and the nitrogen, carbon or sulfur atoms in the heterocydyl radical may be optionally oxidized; the nitrogen atom may be optionally quatemized; and the heterocydyl radical may be aromatic or partially or fully saturated. The heterocydyl radical may not be attached to the rest of the molecule at any heteroatom atom. Examples of such heterocyclyl radicals include, but are not limited to, azepinyl, acridinyl, benzimidazolyl, benzthiazolyl, benzothiadiazolyl, benzoxazolyl, benzodioxolyl, benzodioxinyl, benzopyranyl, benzopyranonyl, benzofuranyl, benzofuranonyl, benzothienyl (benzothiophenyl), benzotriazolyl, carbazolyl, cinnolinyl, decahydroisoquinolyl, dioxolanyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thienyl, thiamorpholinyl, thiamorpholinyl sulfoxide, and thiamorpholinyl sulfone. Unless stated otherwise specifically in the specification, the term “heterocyclyl” is meant to include heterocyclyl radicals as defined above which are optionally substituted by one or more substituents selected from the group consisting of alkyl, alkenyl, aryl, aralkyl, aralkenyl, cycloalkyl, cycloalkylalkyl, halo, haloalkyl, haloalkoxy, nitro, cyano, —R⁵—N═N—O—R⁷, —OR⁶, —C(O)OR⁶, —C(O)N(R⁶)₂, —N(R⁶)₂, —N(R⁶)₂, —N(R⁶)C(O)R⁶, —N(R⁶)C(O)OR⁷, , —C(O)R⁶, —N(R⁶)C(O)N(R⁶)₂, —OC(O)N(R⁶)₂, —N(R⁶)S(O)ₜR⁶ (where t is 0 to 2) —S(O)ₜR⁶ (where t is 0 to 2), —S(O)ₜN(R⁶)₂ (where t is 0 to 2), heterocyclyl and heterocyclylalkyl, wherein each R⁵, R⁶ and R⁷ are as defined above in the Summary of the Invention.

“Heterocyclylalkyl” refers to a radical of the formula —RₐRₑ where Rₐ is an alkyl radical as defined above and Rₑ is a heterocyclyl radical as defined above, and if the heterocyclyl is a nitrogen-containing heterocyclyl, the heterocyclyl may be attached to the alkyl radical at the nitrogen atom. The heterocyclyl radical may be optionally substituted as defined above.

As used herein, compounds which are “commercially available” may be obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Comwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Comwall, U.K.), Lancaster Synthesis (Windham, N.H.), Maybridge Chemical Co. Ltd. (Comwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hannover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified though various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., “Design of Prodrugs” (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Assocation and Pergamon Press, 1987, both of which are incorporated in full by reference herein.

The term “prodrug” is also meant to include any covalently bonded carriers which release the active compound of the invention in vivo when such prodrug is administered to a mammalian subject Prodrugs of a compound of the invention may be prepared by modifying functional groups present in the compound of the invention in such a way that the modifications are cleaved, either in routine manipulation or in vivo, to the parent compound of the invention. Prodrugs indude compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and amine functional groups in the compounds of the invention and the like.

“Stable compound” and “stable structure” are meant to indicate a compound that is sufficiently robust to survive isolation to a useful degree of purity from a reaction mixture, and formulation into an efficacious therapeutic agent.

“Mammal” includes humans and domestic animals, such as cats, dogs, swine, cattle, sheep, goats, horses, rabbits, and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic adds such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, dnnamic add, mandelic acid, methanesulfonic add, ethanesulfonic acid, p-toluenesulfonic acid, salicylic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free add. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“PTPN12” refers to the Human Genome Organization (HUGO) Nomenclature Committee's name for protein tyrosine phosphatase, non-receptor like 12. PTPN12 is also known as PTP-PEST and PTPG1. The coding sequence may be accessed at GenBank; M93425; and is disclosed by Yang et al. (1993) J. Biol. Chem. 268 (9), 6622-6628.

“PTPN2” refers to the Human Genome Organization (HUGO) Nomenclature Committee's name for protein tyrosine phosphatase, non-receptor like 2. PTPN2 is also known as TC-PTP or T-cell-PTP. The sequence of PTPN2 may be accessed at Genbank, M25393, and is described in Cool et al. (1989) Proc. Natl. Acad. Sci. U.S.A. 86 (14), 5257-5261.

“PTPN1” refers to the HUGO Nomenclature Committee's name for protein tyrosine phosphatase, non-receptor like 1. PTPN1 is also known as PTP1B.

PRKD2 refers to the Human Genome Organization (HUGO) Nomenclature Committee's name for protein kinase D2, a human serine threonine protein kinase. PRKD2 is also known as PKD2 or HSPC187. The gene sequence may be accessed at GenBank (accession number NM_{—}016457); and is outlined in Sturany et al., J. Biol. Chem. (2001), Vol. 276, pp. 3310-3318).

GSK3β, refers to the Human Genome Organization (HUGO) Nomenclature Committee's name for glycogen synthase kinase 3-beta. The glycogen-synthase kinase beta was originally cloned from a hepatocarcinoma cell line Hep G2 cDNA library by Stambolic and Woodgeff (Biochem. J. (1994), Vol. 303, pp. 701-704).

“Insulin resistance” includes diabetes, hyperglycemia, and other disorders associated with insulin receptor (IR) signal transduction.

“Therapeutically effective amount” refers to that amount of a compound of formula (I) which, when administered to a mammal, preferably a human, is sufficient to effect treatment, as defined below, for cancer, inflammation, neurological disease or renal disease in the mammal. The amount of a compound of formula (I) which constitutes a “therapeutically effective amount” will vary depending on the compound, the condition and its severity, and the age of the mammal to be treated, but can be determined routinely by one of ordinary skill in the art having regard to his own knowledge and to this disclosure.

“Treating” or “treatment” as used herein covers the treatment of a hyperproliferative disease as disclosed herein, in a mammal, preferably a human, and includes:

(i) preventing cancer, inflammation, diabetes, neurological disease or renal disease from occurring in a mammal, in particular, when such mammal is predisposed to the condition but has not yet been diagnosed as having it;

(ii) inhibiting cancer, inflammation, diabetes, neurological disease or renal disease, i.e., arresting its development; or

(iii) relieving cancer, inflammation, diabetes, neurological disease or renal disease, i.e., causing regression of the condition.

The compounds of formula (I), or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to indude all such possible isomers, as well as, their racemic and optically pure forms. Optically active (+) and (−), (R)- and (S)-, or (D)- and (L)- isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The nomenclature used herein for the compounds of formula (I) is a modified form of the I.U.P.A.C. nomenclature system wherein the compounds are named herein as derivatives of the thiazolidinedithione moiety.

### Methods of Use

This invention is directed to methods of using compounds of formula (I), as set forth above in the Summary of the Invention, and pharmaceutical compositions containing compounds of formula (I) in treating hyperproliferative conditions. Thus, the methods disclosed herein are useful in treating disorders and physiological conditions associated with hyperproliferation and tissue remodelling or repair when administered to a subject in need of such treatment. Of particular interest are hyperproliferative disorders associated with cellular modulation of protein phosphorylation states, i.e. altered activity of phosphorylation modifying enzyme(s), e.g. protein tyrosine kinases and protein tyrosine phosphatases.

In one aspect of the invention, compounds and pharmaceutical compositions of the invention are used to inhibit the activity of PTPN12, PTPN2, PTPN1, PRKD2 and/or GSK3β. These enzymes have been associated with alterations in the phosphorylation state of cellular proteins.

The compounds and pharmaceutical compositions of the invention are administered to a subject having a cancer or a pathological inflammation in order to inhibit tumor growth by impeding cell division, and to decrease inflammation by inhibiting cell adhesion and cell migration. In addition, the methods of the invention may be used in association with restoring the normal foot process architecture of podocytes in glomerular diseases associated with proteinuria (Reiser, J. et al., Rapid Communication, Kidney Int. (2000), Vol. 57, No. 5, pp. 2035-2042).

The compounds and pharmaceuticals compositions of the invention are administered to a subject having diabetes in order to block the insulin-receptor mediated processes influenced by PTPN2 or PTPN1 (Galic, S., Klingler-hofmann, M., Fodero-Tavoletti, M T. et al Mol Cell Biol (2003), Vol. 23, No. 6, pp. 2096-2108; Elchebly, M., Payette, P., Michaliszyn, E., et at Science (1999) Vol. 283, No. 5407, pp.1544-8.)

The methods of the invention can be used prophylactically (i.e., to prevent the disorder of interest from occurring) or therapeutically (i.e., to inhibit or relieve the disorder). As used herein, the term “treating” is used to refer to both prevention of disease, and treatment of pre-existing conditions. The prevention of symptoms is accomplished by administration of the compounds and pharmaceutical compositions of the invention prior to development of overt disease, e.g., to prevent the regrowth of tumors, prevent metastatic growth, diminish restenosis associated with cardiovascular surgery, to prevent or reduce cell migration leading to inflammation and associated tissue damage. Altematively, the compounds and pharmaceutical compositions of the invention may be administered to a subject in need thereof to treat an ongoing disease, by stabilizing or improving the dinical symptoms of the patient

The subject, or patient, may be from any mammalian species, e.g. primates, particularly humans; rodents, including mice, rats and hamsters; rabbits; equines; bovines; canines; felines; etc. Animal models are of interest for experimental investigations, providing a model for treatment of human disease.

Hyperproliferative disorders refers to excess cell proliferation, relative to that occurring with the same type of cell in the general population and/or the same type of cell obtained from a patient at an earlier time. The term denotes malignant as well as non-malignant cell populations. Such disorders have an excess cell proliferation of one or more subsets of cells, which often appear to differ from the surrounding tissue both morphologically and genotypically. The excess cell proliferation can be determined by reference to the general population and/or by reference to a particular patent, e.g. at an earlier point in the patient's life. Hyperproliferative cell disorders can occur in different types of animals and in humans, and produce different physical manifestations depending upon the affected cells.

Hyperproliferative cell disorders include cancers; blood vessel proliferative disorders such as restenosis, atherosclerosis, in-stent stenosis, vascular graft restenosis, etc.; fibrotic disorders; psoriasis; inflammatory disorders, e.g. arthritis, etc.; glomerular nephritis; endometriosis; macular degenerative disorders; benign growth disorders such as prostate enlargement and lipomas; and autoimmune disorders. Cancers of particular interest include carcinomas, e.g. colon, prostate, breast, melanoma, ductal, endometrial, stomach, dysplastic oral mucosa, invasive oral cancer, non-small cell lung carcinoma, transitional and squamous cell bladder carcinoma, etc.; neurological malignancies, e.g. neuroblastoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; sarcomas, melanomas, adenomas; benign lesions such as papillomas, and the like.

Other hyperproliferative disorders that may be associated with altered activity of phosphorylation modifying enzyme(s) include a variety of conditions where there is proliferation and/or migration of smooth muscle cells, and/or inflammatory cells into the intimal layer of a vessel, resulting in restricted blood flow through that vessel, i.e. neointimal occlusive lesions. Occlusive vascular conditions of interest include atherosclerosis, graft coronary vascular disease after transplantation, vein graft stenosis, peri-anastomatic prosthetic graft stenosis, restenosis after angioplasty or stent placement, and the like.

Disorders and conditions where there is hyperproliferation and/or tissue remodelling or repair of reproductive tissue, e.g. uterine, testicular and ovarian carcinomas, endometriosis, squamous and glandular epithelial carcinomas of the cervix, etc. are reduced in cell number by administration of the compounds and pharmaceutical compositions of the invention. Other disorders and conditions of interest relate to epidermal hyperproliferation, tissue remodelling and repair. For example, the chronic skin inflammation of psoriasis is associated with hyperplastic epidermal keratinocytes.

Other disorders of interest include inflammatory disorders and autoimmune conditions including, but not limited to, psoriasis, rheumatoid arthritis, multiple sclerosis, scleroderma, systemic lupus erythematosus, Sjögren's syndrome, atopic dermatitis, asthma, and allergy. Target cells susceptible to the treatment include cells involved in instigating autoimmune reactions as well as those suffering or responding from the effects of autoimmune attack or inflammatory events, and include lymphocytes and fibroblasts.

The susceptibility of a particular cell to treatment according to the invention may be determined by in vitro testing. Typically, a culture of the cell is combined with a subject compound at varying concentrations for a period of time sufficient to allow the active agents to induce cell death or inhibit migration, usually between about one hour and one week. For in vitro testing, cultured cells from a biopsy sample may be used.

The dose will vary depending on mode of administration, specific disorder, patient status, etc. Typically a therapeutic dose will be sufficient to substantially decrease the undesirable cell population in the targeted tissue, while maintaining patient viability. Treatment will generally be continued until there is a substantial reduction, e.g. at least about 50%, decrease in the clinical manifestation of disease, and may be continued until there are essentially none of the undesirable cellular activity detected in the relevant tissue.

The compounds of formula (I) may also find use in the specific inhibition of signaling pathways mediated by protein tyrosine phosphatases, for example, PTPN12 and PTPN2, and as a “positive” control in high throughput screening for other modulating compounds. In particular, this invention directed to methods of using compounds of formula (I) and pharmaceutical compositions containing such compounds in treating cancer, diabetes or inflammation associated with PTPN12, PTPN2, PRKD2 and/or GSK3β activity.

PTPN12 contains a proline rich motif at its C-terminal and can bind to p130^{cas}, which is a focal adhesion associated protein containing an SH3 domain. In normal cells, p130^{cas} becomes highly phosphorylated following integrin dependent activation of the fak and src kinases. This phosphorylation appears to allow a series of tyrosine dependent signalling that has among other consequences the actin filament reorganization. Because of the importance of integrin signalling in the cell cytoskeleton, motility and transformation, the action of PTPN12 on p130^{cas} may have dramatic consequences in mammalian development as well as in some physiopathological events. The process of cell migration is crucial for the correct development of a mammalian embryo. In an adult organism, cell migration plays an important role in events like invasion of a wounded space by fibroblasts and endothelial cells and translocation of lymphocytes and neutrophiles to an inflammation site. In cancer, tumor cells also have to migrate in order to reach the circulatory system and disperse throughout the organism. Takekawa, M. et al, FEBS Lett.(1994), Vol. 339, pp. 222-228 discloses aberrant transcripts of PTPN12 in cancer cells. The effect of PTPN12 levels on fibroblast motility is described in Garton et al. (1999) J. Biol. Chem. 274(6):3811-3818. Davidson et al. (2001) EMBO. J. 20(13):3414-26 discusses a connection of PTPN12 with inflammation. The relationship between PTPN12 and podocyte regulation in kidney is described in Reiser, J. et al., Rapid Communication, Kidney Int. (2000), Vol. 57, No. 5, pp. 2035-2042.

PTPN12 is involved in signalling pathways for such important cellular activities as responses to extracellular signals and cell cycle checkpoints. Inhibition of PTPN12 provides a means (for example, by blocking the effect of an extracellular signal) of intervening in these signalling pathways, which are associated with a variety of pathological or clinical conditions. PTPN12 is associated with cell adhesion, cell division and cell migration and thus is implicated in cancer and inflammation.

Another PTP of particular interest is PTPN2. PTPN2 is also known as T-cell protein tyrosine phosphatase (TC-PTP) and was first identified by Cool et al., Proc. Natl. Acad. Sci. (1989), Vol. 86, pp.5257-5261. PTPN2 exists in two forms generated by altemative splicing: a 48kDa endoplasmic reticular (ER)-associated form called TC48 (PTP-S4); and a 45-kDa nuclear form called TC45 (PTP-S2). PTPN2 plays a significant role in both hematopoiesis and immune function. You-Ten et al., J. Exp. Med. (1997), Vol.186, No. 5, pp. 683-693 found that PTPN2 −/− mice die between 3-5 weeks of age, exhibiting specific defects in bone marrow (BM), B cell lymphopoeisis, and erythropoiesis, as well as impaired T and B cell functions. B.M. transplantation experiments demonstrated that hematopoietic failure in the homozygotes was not due to a stem cell defect but rather stromal cell deficiency.

PTPN2 may play a role in cancer progression and metastases. Mitra, S. K. et al., Exp. Cell Res.15 (2001), Vol. 270, No. 1, pp. 32-44 demonstrated inhibition of anchorage-independent cell growth, adhesion, and cyclin D1 gene expression by a dominant negative mutant PTPN2. Expression of mutant PTPN2 in PyF cells resulted in strong inhibition of anchorage-independent growth in soft agar but had no significant effect on growth in liquid culture. Tumor formation in nude mice was also reduced by the presence of mutant PTPN2.

PTPN2 plays a role in apoptosis, making it a useful target for cancer therapy or as a component of a cancer therapeutic cocktail. Zsigmond, E. et al, FEBS Lett. (1999), Vol. 453, No. 3, pp. 308-312, found that overexpression of PTPN2 induced nuclear fragmentation typical of apoptosis. In addition, PTPN2 appears to be active in progressing the early G1 phase of the cell cycle through the NF-kappaB pathway (Ibarra-Sanches, M. J. et al., Oncogene (2001), Vol. 20, No. 34, pp. 4728-39). Inhibition of PTPN2 is useful in treating conditions associated with PTPN2 activity, such as inflammation, cancer progression and metastases.

PTPN2 isoforms TC45 and TC48 have also been studied in association with insulin receptor signaling, and results suggest that insulin receptor may act as a substrate for PTPN2, and that the interaction of PTPN2 and IR may result in downregulatin of insulin-induced signaling in vivo (Galic, S., Klingler-hofmann, M., Fodero-Tavoletti, M T. et al Mol Cell Biol (2003), Vol. 23, No.6, pp. 2096-2108).

PTPN1 activity is associated with insulin resistance, and diabetes, hyperglycemia, and other disorders associated with insulin receptor (IR) signal transduction (Elchebly, M., Payette, P., Michaliszyn, E., et al. Science (1999) Vol. 283, No. 5407, pp.1544-8). Reduction of PTPN1, for example, is sufficient to increase insulin-dependent metabolic signaling and improve insulin sensitivity (Gum, R. J.; Gaede, L. L.; Koterski, S. L. et al., Diabetes (2003) 52(1):21-8). When PTPN1 is overexpressed, it plays a role in insulin resistance (Ahmad, F. et al.,(1997) J. Clin. Invest. 100: 449-458).

Another protein tyrosine kinase of particular interest is protein kinase D2, also known as PRKD2. PRKD2 is a human serine threonine protein kinase gene (GenBank accession number NM_{—}016457; Sturany et al., J. Biol. Chem. (2001), Vol. 276, pp. 3310-3318). The protein sequence contains two cysteine-rich motifs at the N terminus, a pleckstrin homology domain, and a catalytic domain containing all the characteristic sequence motifs of serine protein kinases. It exhibits the strongest homology to the serine threonine protein kinases PKD/PKCμ and PKC, particularly in the duplex zinc finger-like cysteine-rich motif, in the pleckstrin homology domain and in the protein kinase domain. The mRNA of PRKD2 is widely expressed in human and murine tissues. Dot blot analysis of probes prepared from mRNA of tumors showed that expression of PRKD2 is consistently up-regulated in clinical samples of human tumors. Significant increases in gene expression for a number of tumor types have been observed by others (Su et al., Cancer Research (2001), Vol. 61, pp. 7388-7393). PRKD2 inhibitors are effective in treating certain types of cancer.

A third PTK of interest relating to this invention is glycogen synthase kinase-3 (GSK3), a proline-directed serine-threonine kinase that was initially identified as a phosphorylating and inactivating glycogen synthase. Two isoforms, alpha (GSK3A; 606784) and beta, show a high degree of amino acid homology (Stambolic, V. et al., “Mitogen inactivation of glycogen synthase kinase-3 beta in intact cells via serine 9 phosphorylation”, Biochem. J. (1994), Vol. 303, pp. 701-704). GSK3β, is involved in energy metabolism, neuronal cell development, and body pattem formation (Plyte, S. E. et al.; “Glycogen synthase kinase-3: functions in oncogenesis and development”, Biochim. Biophys. Acta (1992), Vol. 1114, pp. 147-162).

Lucas et al. generated mice overexpressing GSK3β, in the brain during adulthood (Lucas, J. J. et al., “Decreased nuclear beta-catenin, tau hyperphosphorylation and neurodegeneration in GSK-3-beta conditional transgenic mice”, EMBO J. (2001), Vol. 20, pp. 27-39). These mice showed decreased levels of nuclear beta-catenin (116806) and hyperphosphorylation of tau (157140) in hippocampal neurons, the latter resulting in pretangle-like somatodendritic localization of tau. Reactive astrocytosis and microgliosis were also indicative of neuronal stress and cell death, which was confirmed by TUNEL assay. Lucas et al. concluded that in vivo overexpression of GSK3β results in neurodegeneration and suggested that these mice can be used as an animal model to study the relevance of GSK3β deregulation to the pathogenesis of Alzheimer's disease.

In one embodiment of the invention, methods are provided for using compounds of formula (I) and pharmaceutical compositions containing such compounds in treating hyperproliferative disorders. Thus, the methods disclosed herein are useful in treating disorders and physiological conditions associated with hyperproliferation and tissue remodeling or repair when administered to a subject in need of such treatment. The compounds and pharmaceutical compositions of the invention are administered to a subject having a cancer or a pathological inflammation in order to inhibit tumor growth by impeding cell division, and to decrease inflammation by inhibiting cell adhesion and cell migration. In addition, the methods of the invention may be used in association with restoring the normal foot process architecture of podocytes in glomerular diseases associated with proteinuria (Reiser, J. et al., Rapid Communication, Kidney Int. (2000), Vol. 57, No. 5, pp. 2035-2042), or reducing the symptoms of diabetes (Galic, S., Klingler-hofmann, M., Fodero-Tavoletti, M T. et al. Mol Cell Biol (2003), Vol. 23, No. 6, pp. 2096-2108; Elchebly, M., Payette, P., Michaliszyn, E., et al. Science (1999) Vol. 283, No. 5407, pp.1544-8.)

The compounds of formula (I) may also find use as affinity reagents for the isolation and/or purification of phosphatases using the biochemical affinity of the enzyme for inhibitors that act on it. The compounds are coupled to a matrix or gel. The coupled support is then used to separate the enzyme, which binds to the compound, from a sample mixture, e.g., a cell lysate, which may be optionally partially purified. The sample mixture is contacted with the compound coupled support under conditions that minimize non-specific binding. Methods known in the art include columns, gels, capillaries, etc. The unbound proteins are washed free of the resin and the bound proteins are then eluted in a suitable buffer.

The compounds of formula (I) may also be useful as reagents for studying signal transduction or any of the clinical disorders listed throughout this application, and for use as a positive control in high throughput screening.

### Administrattio of the Compounds and Pharmaceutical Composltions of the Invention

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrastemal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disorder or condition associated with hyperproliferation and tissue remodelling or repair in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol; which is useful in, e.g., inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel™, corn starch and the like; lubricants such as magnesium stearate or Sterotex™; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or a fatty oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 1% by weight of the compound of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Altematively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

Whether in solid, liquid or gaseous form, the pharmaceutical composition of the present invention may contain one or more known pharmacological agents used in the treatment of cancer or inflammation in a mammal, particularly, cancer or inflammation associated with hyperproliferation and tissue remodelling or repair.

The pharmaceutical compositions of the invention may be prepared by methodology well known in the pharmaceutical art. For example, a pharmaceutical composition intended to be administered by injection can be prepared by combining a compound of the invention with water so as to form a solution. A surfactant may be added to facilitate the formation of a homogeneous solution or suspension. Surfactants are compounds that non-ovalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is from about 0.1 mg to about 20 mg/kg of body weight per day of a compound of the invention, or a pharmaceutically acceptable salt thereof; preferably, from about 0.1 mg to about 10 mg/kg of body weight per day; and most preferably, from about 0.1 mg to about 7.5 mg/kg of body weight per day.

### PREFERRED EMBODIMENTS OF THE INVENTION

Of the various methods set forth above in the Summary of the Invention, preferred methods are those methods wherein the compound of formula (I) is a compound of formula (Ia):

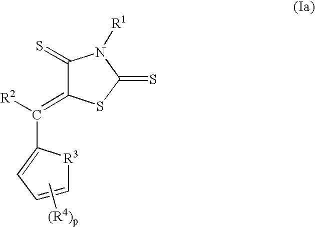

wherein:

p is 0 to 3;

R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;

R² is hydrogen, alkyl, aralkyl, aryl, haloalkyl, cycloalkyl, cydoalkylalkyl, heterocyclyl or heterocyclylalkyl;

R³ is —O— or —S—;

each R⁴ is independently selected from the group consisting of alkyl, alkenyl, aryl, aralkyl, aralkenyl, cydoalkyl, cycloalkylalkyl, halo, haloalkyl, haloalkoxy, nitro, cyano, —R⁸—N═N—O—R⁷, —OR⁶, —C(O)OR⁶, —C(O)N(R⁸)₂, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —C(O)R⁶, —N(R⁶)C(O)N(R⁶)₂, —OC(O)N(R⁶)₂, or —N(R⁶)S(O)ₜR⁶ (where t is 0 to 2) heterocyclyl and heterocyclylalkyl;

each R⁵ is independently an optionally substituted straight or branched alkylene or alkenylene chain;

each R⁶ is independently hydrogen, alkyl, alkenyl, cycloalkyl, cycloalkylalkyl, aralkyl or aryl;

each R⁷ is independently hydrogen, alkyl or aralkyl; and

R⁸ is a direct bond or an optionally substituted straight or branched alkylene or alkenylene chain.

Of these preferred methods, a preferred group of methods is that group of methods to treat cancer, diabetes or inflammation in a mammal wherein the mammal is human. Of this preferred group, a preferred subgroup of methods is that subgroup wherein the cancer or inflammation is associated with hyperproliferation or tissue remodelling or repair. Of this preferred subgroup, a preferred class of methods is that class wherein the cancer or inflammation is associated with the activity of an enzyme selected from the group consisting of PTPN12, PTPN2, PRKD2, and GSK3β.

Of this preferred group, subgroup and class of methods set forth above, a preferred subclass of methods is that subclass wherein the compound of formula (Ia) is a compound of formula (Ia) wherein:

p is 1;

R¹ is hydrogen, alkyl, or aralkyl;

R² is hydrogen or alkyl;

R³ is —O— or —S—; and

R⁴ is halo, haloalkyl, or haloalkoxy.

Of the preferred methods of the invention as set forth above, another preferred group of methods is that group of methods to treat a mammalian cell with a compound of formula (Ia) wherein the mammalian cell is treated in vitro. Another preferred group of these preferred methods is that group of methods to treat a mammalian cell with a compound of formula (Ia) wherein the mammalian cell is treated in vivo. Another preferred group of these preferred methods is that group of methods to treat a mammalian cell with a compound of formula (Ia) wherein the inhibition of activity of PTNP12, PTPN2, PRKD2 and/or GSK3β results in a reduction of cell adhesion. Another preferred group of these of these preferred methods is that group of methods to treat a mammalian cell with a compound of formula (Ia) wherein the inhibition of activity of PTNP12, PTPN2, PRKD2 and/or GSK3β results in a reduction of cell division. Another preferred group of these of these preferred methods is that group of methods to treat a mammalian cell with a compound of formula (Ia) wherein the inhibition of activity of PTNP12, PTPN2, PRKD2 and/or GSK3β results in a reduction of cell migration. Another preferred group of these of these preferred methods is that group of methods to treat a mammalian cell with a compound of formula (Ia) wherein the inhibition of activity of PTNP12, PTPN2, PRKD2 and/or GSK3β results in control of tumor growth. Another preferred group of these of these preferred methods is that group of methods to treat a mammalian cell with a compound of formula (Ia) wherein the inhibition of activity of PTNP12, PTPN2, PRKD2 and/or GSK3β results in control of lymphocyte activation.

Another preferred group of methods is that group of methods to treat a mammalian cell with a compound of the formula (I) or (Ia) wherein the inhibition of activity of PTPN2 and/or PTPN1 results in control of disease mediated by the insulin receptor.

Of the pharmaceutical compositions of the invention as set forth above in the Summary of the Invention, preferred pharmaceutical compositions are those pharmaceutical compositions wherein the compound of formula (Ia) is a compound of formula (Ia) wherein:

p is 1;

R¹ is hydrogen, alkyl, or aralkyl;

R² is hydrogen or alkyl;

R³ is —O— or —S—; and

R⁴ is halo, haloalkyl, or haloalkoxy.

### Preparation of the Compounds of Formula (I) and Formula (IA)

Compounds of formula (I) and formula (Ia) in the methods and pharmaceutical compositions of the invention may be prepared according to methods known to one skilled in the art, or by the methods similar to those disclosed in Ead, H. A., et al., Arch. Pharmacal Res. (1990), Vol. 13, No. 1, pp. 5-8, or by methods similar to the method described below.

It is understood that in the following description, combinations of substituents and/or variables of the depicted formulae are permissible only if such contributions result in stable compounds.

It will also be appredated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyidiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters.

Protecting groups may be added or removed in accordance with standard techniques, which are well-known to those skilled in the art and as described herein.

The use of protecting groups is described in detail in Green, T. W. and P. G. M. Wutz, Protective Groups in Organic Synthesis (1991), 2nd Ed., Wiley-Interscience. The protecting group may also be a polymer resin such as a Wang resin or a 2-chlorotrityl chloride resin.

It will also be appreciated by those skilled in the art, although such protected derivatives of compounds of formulae (I), as described above in the Summary of the Invention, may not possess pharmacological activity as such, they may be administered to a mammal with cancer or inflammation and thereafter metabolized in the body to form compounds of the invention which are pharmacologically active. Such derivatives may therefore be described as “prodrugs”. All prodrugs of compounds of formula (I) are included within the scope of the invention.

For illustration purposes only, the following Reaction Scheme depicts the preparation of compounds of formula (Ia) where R¹, R², R³ and R⁴ are as described above in the Preferred Embodiments. It is understood, however, that other compounds of formula (I) may be prepared in a similar manner by methods known to one skilled in the art.

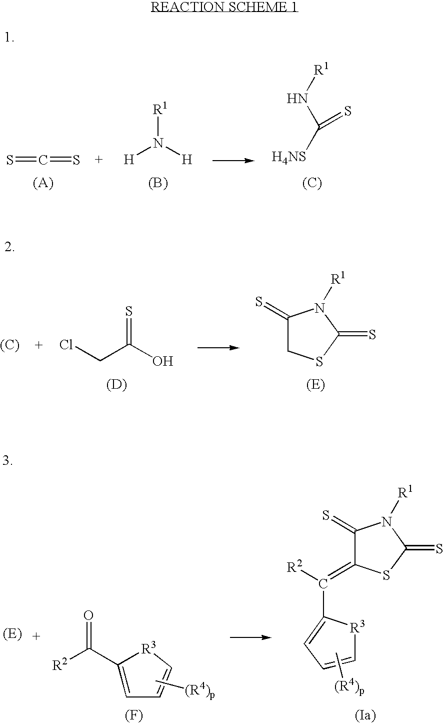

In this general scheme, starting components may be obtained from sources such as Aldrich, or synthesized according to sources known to those of ordinary skill in the art, e.g., Smith and March, March's Advanced Organic Chemistry: Reactions, Mechanisms, and Structure, 5^{th} edition (Wiley Interscience, New York). Groups R¹, R², R³, and R⁴ are selected from components as defined in the specification heretofore.

In general, dithiocarbamate compounds of formula (C) may be prepared by reacting carbon disulfide (i.e., the compound of formula (A)) at a concentration of about 3.5 moles/liter, with an amine compound of formula (B) at a concentration of about 2.8 moles/liter, in the presence of ammonium hydroxide solution at about 0° C. The admixture is warmed to ambient temperature, stirred for up to about 18 hours, and concentrated to dryness. The resulting substance is a compound of formula (C).

Compounds of formula (D) wherein may be obtained from a source such a Aldrich, or prepared according to schemes known to those of ordinary skill in the art. For instance, a hydroxycarbonylmethylenehalide compound may be reacted with about an equimolar amount of diphosphorus pentasulfide to afford a hydroxythiocarbonylmethylenehalide compound of formula (D), which can then be used in the reaction scheme as set forth heretofore.

Rhodanine-derived compounds of formula (E) can be prepared under cyclization conditions according to schemes known to those of ordinary skill in the art. See, for example, Ead, H. A. et al., Arch. Pharmacal. Res. (1990), Vol 13, No. 1, pp. 5-8. For instance, a compound of formula (E) is formed by combining the foregoing quantity of the compound of formula (C) with a compound of formula (D) or a basic salt thereof, at a concentration of about 3 moles/liter, in an aqueous solution (at about 0° C.) alkalized with dilute sodium carbonate. The reaction mixture is warmed to ambient temperature, admixed with about 6.4 volumes of warm 5 M hydrochloric acid (about 70° C.), and heated to about 90° C. for about 1 hour. After cooling, the resulting precipitate is isolated by filtration, washed with water and allowed to dry, affording a compound of formula (E).

Substituted heteroaryl compounds of formula (F) (wherein R³ is O or S) can be obtained from sources such as Aldrich, or prepared according to schemes known to those of ordinary skill in the art. In one aspect, nitro-substituted compounds of formula (F) may be prepared under standard electrophilic aromatic substitution conditions, such as by treatment of 2-furancarboxaldehyde or 2-thiophenecarboxaldehyde with nitric acid and sulfuric acid. In another aspect, halogen-substituted compounds of formula (F) may be prepared under standard electrophilic aromatic substitution conditions, such as by treatment of 2-furancarboxaldehyde or 2-thiophenecarboxaldehyde with naturally-occurring diatomic halogen compounds (i.e., F₂, Cl₂, Br₂, or I₂) in the presence of iron metal. In yet another aspect, alkyl-substituted compounds of formula (F) may be prepared under standard alkylation conditions, such as by Friedel-Crafts alkylation of 2-furancarboxaldehyde or 2-thiophenecarboxaldehyde with an alkyl halide in the presence of an aluminum halide compound; or by Friedel-Crafts acylation of 2-furancarboxaldehyde or 2-thiophenecarboxaldehyde with an acyl halide in the presence of an aluminum halide or stannic halide compound, followed by reduction under standard conditions. Such treatments normally produce mixtures comprising compounds with substitutions in various different ring positions, though specific chemical properties of the reagents used, particularly the aromatic compound, may promote the synthesis of certain compounds with substitutions at specified ring positions as major synthesis products. Collection of pure major and/or minor synthesis products may be achieved with the use of a preparative separation and isolation technique such as high performance liquid chromatography (HPLC).

Compounds of formula (Ia) can be prepared under standard condensation reaction conditions according to schemes known to those of ordinary skill in the art. For instance, a compound of formula (I) is formed by combining a rhodanine-derived compound of formula (E), at a concentration of about 0.5 to 2.0 moles/liter, with about an equimolar quantity of a substituted heteroaryl compound of formula (F) (wherein R²,R³, and R⁴ are selected from constituents as defined in the specification) in an aqueous solution containing sodium acetate (about 1 to 6 moles/liter) and glacial acetic acid. The reaction is heated to reflux for up to 16 hours with stirring. After cooling and optional addition of up to about 7 volumes of water, the resulting precipitate is isolated by filtration, washed with water and allowed to dry, affording a compound of formula (Ia) and/or a stereoisomer of the compound of formula (I), and/or salt(s) of the compound(s) thereof.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXAMPLE 1

### Enzyme Preparation and Use

A. PTPN2

PTPN2 was cloned from a human placental cDNA library in the IMPACT™ (New England BioLabs) bacterial expression system. The technology was first described by Chong et al., Gene (1997), Vol. 192, pp. 271-281. The IMPACT™ Protein Purification System was purchased commercially from New England BioLabs. The resulting product was used in the development of a protein phosphatase assay for high-throughput screening (HTS) of target molecules and in other assays described herein (see Example 2).

Biochemical analysis performed on recombinant human PTPN2 fusion protein exhibited protein phosphatase activity in the order of 1500 to 2500 pmol/min/μg measured as phosphate release from a synthetic tyrosine phosphorylated peptide. This activity was considered to be in the high range as compared to other recombinant protein tyrosine phosphatases assayed. PTPN2 preparations were subsequently used extensively in in vitro assays for the initial discovery of compounds having the ability to inhibit PTPN2 activity.

B. PTPN12

PTPN12 was cloned in the IMPACT™ (New England BioLabs) bacterial expression system. The IMPACT™ Protein Purification System was purchased commercially from New England BioLabs.

1. Cloning of Truncated Human PTPN12 into pTWIN-II Expression Vector

Expression of human truncated PTPN12 as a fusion protein required that the cDNA be ligated into the polyclonal site situated in frame and upstream of the intein gene of the IMPACT™ expression vector pTWIN-II. The truncated version was used as it was far easier to handle and gave parallel results to the full length protein in comparison testing. For the purpose of simplicity, PTP-PEST-N will be used interchangeably with PTPN12 in these Examples.

The PTPN12 coding sequence was generated by polymerase chain reaction (PCR) using gene-specific primers.

2. Human PTPN12 Expression and Purification

Active PTPN12 enzyme is expressed from the IMPACT™ vector system in the bacterial strain ER2566. Recombinant PTPN12 protein is purified from bacterial cells using affinity chromatography on chitin-agarose beads followed by a chemical process whereby PTPN12 is released from its affinity tag. A complete quantitative and qualitative analysis of the protein is monitored using Coomassie-Blue staining of SDS-PAGE separated preparations and by PTPN12-specific westem blotting. PTPN12 is produced at levels in the range of 0.1-0.5 mg per litre of bacterial cell culture.

3. PTPN12 In Vitro Phosphatase Assay

Biochemical analysis is performed on recombinant human PTPN12 fusion protein. Typically, the PTPN12 preparations are found to exhibit protein phosphatase reactivity in the order of 1500 to 2500 pmol/min/μg measured as phosphate release from a synthetic tyrosine phosphorylated peptide. This activity is considered to be in the high range as compared to other recombinant protein tyrosine phosphatases. PTPN12 preparations were subsequently used extensively in in vitro assays for the initial discovery of compounds having the ability to inhibit PTPN12 activity.

### EXAMPLE 2

### In Vitro Activity Profile for Phosphatases

Compounds of formula (I) and formula (Ia) were tested in the following assay for their ability to inhibit the activity of the desired phosphatase.

A. Reagent Preparation:

1. Malachite Green-Ammonium Molybdate Reagent

Two solutions were first prepared. Solution 1 contained 4.2% ammonium molybdate tetrahydrate (Sigma, Cat# A-7302) in 4 N HCl. Solution 2 contained 0.045% Malachite Green (Sigma, Cat. # M-9636). The two solutions were mixed as follows: 250 mL of solution 1 and 750 mL solution 2 with constant stirring for 20 min. The resulting mixture was filtered through 0.22 μM filter (one can use Nalgene™ bottle top vacuum filters Cat # 28199-317). The solution was stored in a brown bottle at 4° C.

B. Preparation of 1 mM ppC SRC 60 Substrate

The peptide sequence: TSTEPQY(PO₄)QPGENL was prepared by conventional methods. Of this,154 mg was dissolved in 100 mL dH₂O and the solution vortexed until the peptide dissolved completely. The ppC SRC 60 was then stored in 1 mL aliquots at −20° C. This is the “Substrate” used for preparing the substrate working stock solution.

C. Procedure for Assay

The enzyme (phosphatase) activity was determined in a reaction that measured phosphate relase from tyrosine phospho-specific peptides using a method first described by Harder et al., Biochem. J. (1994), Vol. 298, pp. 395-401. This is a non-radioactive method for measuring free phosphate by the malachite green method first described by Van Veldhoven and Mannaerts, Anal Biochem. (1987), Vol.161, pp.45-48. 10×assay buffer (250 mM Tris:100 mM, β-mercaptoethanol, 50 mM EDTA; pH 7.2) was diluted to 5×concentration (conc.) with distilled H₂O (dH₂O). Then 71.4 μM of substrate working stock solution was prepared in dH₂O.

In a microcentrifuge tube, the required volume. of enzyme stock was pipetted, diluted with the required volume of 5×assay buffer and mixed.

The colour reagent was prepared by thoroughly mixing 10 mL malachite green-ammonium molybdate reagent and 100 μL of 1% Tween-20 (1 mL Tween-20 (BDH, #06435) dissolved in 99 mL dH₂O) into a reagent reservoir and stored at room temperature. Approximately 10 mL of colour reagent is required per assay plate, or 100 μL per well.

Sample Compound Preparation

In a Falcon™ 96 well plate the sample compound was diluted in 1% DMSO (1 mL DMSO (Sigma, Cat. # D-8779) dissolved in 99 mL dH₂O and stored at room temperature) such that the concentration of the sample compound working stock solution is ten times the final desired concentration of the compound in the assay.

The working stock solution was prepared as per the required concentration of sample compound in the assay.

The negative control consisted of 5 μl 1% DMSO and 35 μL substrate working stock solution and 10 μL diluted enzyme, per well, and was placed in the first column of wells on the plate. The last column of wells on the plate was reserved for an enzyme blank, which consisted of 5 μL 1% DMSO, 35 μL substrate working stock solution, and 10 μL 5×assay buffer, per well. Test samples were placed in columns 2-11 and consisted of 5 μL sample in 1% DMSO, 35 μL substrate working stock solution, and 10 μL of diluted enzyme, per well, at the desired concentration. Using the repeater function of a Biohit Multichannel™ pipettor, 5μL of 100 μM sample from the Falcon™ plate columns was added to corresponding Costar™ assay plate columns.

Then 5 μL 1% DMSO was added to column 1 & 12, and 10 μL of 5×assay buffer to column 12.

Using a multichannel pipettor, 35 μL of 71.4 μM ppC-SRC 60 substrate was added to all assay wells, then 10 μL of appropriately diluted enzyme was added to the wells on a column by column basis, pausing 5 seconds between columns. Timing started at the first addition.

The assay plate was incubated at room temperature (21° C.) for 15 minutes. The reaction was “stopped” by adding 100 μL color reagent on a column by column basis, pausing 5 seconds between columns. Color was allowed to develop for at least 15 minutes, but no longer than two hours, at room temperature. The plate was “read” on Bio-tek Instruments EL312e™ microplate Bio-Kinetics™ reader at 590 nm and the data collected as per instrument manual.

Data analysis was performed as follows. The blank and negative controls were read, and blanks were subtracted from the average of negative control values and sample values, and the % inhibition was expressed by the following formula:
% Inhibition=100−[corrected sample reading/corrected Negative Control reading*100].

Compounds of the invention showed the following profile of inhibition:

TABLE 1

| | Inhibition of | Inhibition of | Inhibition of |
|---|---|---|---|
| Compound | PTPN12 IC₅₀ | PTPN2 IC₅₀ | PTPN1 IC₅₀ |
| 5-[5-(4-Bromophenyl)- | 0.7 | 2.1 | 5.2 |
| furan-2-ylmethylene]- | | | |
| thiazolidine-2,4-dithione | | | |

### EXAMPLE 3

### PRKD2 Preparation and Assay

A. Enzyme Preparation:

The PRKD2 cDNA (Kinetek clone # K237) was amplified from human brain CDNA library by polymerase chain reaction (PCR) and cloned directly into pPCR-Script Amp SK(+) cloning vector (Strategene). After DNA sequendng, the confirmed 2637 bp human PRKD2 cDNA was subcloned into baculovirus expression vector pAcG4T3. This vector was created from pAcG2T (BD Pharmingen) by adding additional restriction sites to the multiple cloning site region. Subsequently, pAcG4T3 was used for recombinant protein expression in insect cells and development of a protein kinase assay for HTS.

Expression of human PRKD2 as a fusion protein required that the cDNA be ligated into the polyclonal site situated in frame and downstream of the glutathione-S-transferase gene of the baculovirus expression vector pAcG4T3. The PRKD2 coding sequence was amplified by polymerase chain reaction using gene-specific forward and reverse oligonucleotide primers pattemed after the 5′ and 3′ coding regions of the original sequence. The PRKD2 cDNA was cloned into vector pPCR-Script Amp SK(+) using PCR-Script Amp™ Cloning Kits included Pfu DNA polymerase, SrfI restriction enzyme and T4 DNA ligase (Strategene). Sequence analysis of the amplified clone revealed no discordance with the original wild type sequence.

The pPCR-Script Amp SK(+)-PRKD2 done was digested with BamHI and XhoI restriction enzymes and the fragment was subsequently cloned into the same sites of pAcG4T3. Confirmation of PRKD2 insertion in the pAcG4T3 vector was determined by restriction analysis.

Active human GST-fusion PRKD2 enzyme was expressed using the baculovirus expression vector system. Infectious baculovirus was generated by co-transfecting recombinant pAcG4T3-PRKD2 with linear AcNPV (Autographa californica nuclear polyhedrosis virus) DNA (BD Pharmingen) into adherent spodoptera fugiperda Sf9 insect cells (Invitrogen) using the protocol provided by the manufacturer. Recombination between homologous sites allowed the heterologous GST-PRKD2 gene transfer from the transfection vector pAcG2T-PRKD2 to the genomic AcNPV DNA and finally the production and amplification of packaged baculovirus particles.

The recombinant GST-PRKD2 protein was produced in High Five™ cells (Invitrogen) in suspension expression conditions, and purified on GST-glutathione affinity system. After 72 hours of expression, the cells were centrifuged and the pellet was lysed in lysis buffer (50 mM Tris-HCL, pH 7.5, 2.5 mM EDTA, 150 mM NaCl, 1% NP-40, 0.1% β-mercaptoethanol, 0.5 mM sodium orthovanadate, 50 mM β-glycerophosphate, 0.1 mM PMSF, 1 mM benzamidine and 0.5% (V/V) protease inhibitor cocktail set III (CalBiochem)). The lysate was cleared of cellular debris by centrifutation and the cleared supemantant was incubated with glutathione beaded agarose (Sigma) by batch-bind rotation according to the manufacturer's instructions (Pharmacia). Following batch binding of the fusion proteins to glutathione-agarose beads, the matrix was transferred to a 1×10 cm Flex-column™ (Kontes Glass) chromatography system. The column was washed with high-salt buffer (50 mM Tris-HCl, pH 7.5, 1 mM EDTA, 500 mM NaCl, 0.1% NP-40, 0.1% β-mercaptoethnaol, 0.5 mM sodium orthovanadate, 50 mM ⊕-glycerophosphate, 0.1 mM PMSF and 1 mM bezamidine). Finally, the GST-PRKD2 protein was eluted from the column using a reduced glutathione buffer (50 mM Tris-HCl, pH7.5, 50 mM NaCl, 10 mM glutathione, 0.1% β-mercaptoethanol and 1 mM PMSF). A complete quantitative and qualitative analysis of the protein was monitored using Coomassie blue staining and GST-specific Western blotting (Kinetek).

B. PRKD2 In Vitro Kinase Assay:

Biochemical analysis was performed on recombinant human GST-PKD2 fusion protein using the experimental protocol outlined in the section entitled “IN VITRO ACTIVITY PROFILE FOR KINASES” infra. Generally, the GST-PKD2 preparations are found to exhibit good protein phosphotransferase activity in the order of 150 pmol/min/μg in the presence of 50 uM of [γ-³²P]-ATP and 116.5 uM of substrate CREBtide™ peptide (amino acid sequence: KRREILSRRPSYR) during a 15 min reaction at ambient temperature.

### EXAMPLE 4

### GSK3-Beta Preparation and Assay

A. Enzyme Preparation:

The original pBluescript-SK-h-GSK3β form was a generous gift from Dr. James Woodgett's laboratory at the Ontario Cancer Institute (Biochem. J. (1994), Vol 303, pp. 701-704). Expression of human GSK3β as a fusion protein required that the cDNA be ligated into multiple cloning sites in frame and downstream of the glutathione-S-transferase gene of the bacterial expression vector pGEX-4T3 (Pharmacia). The GSK3β coding sequence was amplified by polymerase chain reaction using gene-specific forward and reverse oligonucleotide primers complementary to the 5′ and 3′ coding regions of the original pBluescript-SK-GSK3β clone. Additional bases were inserted at the 5′ end of each DNA primer to facilitate sub-cloning and expression of the amplified CDNA product into the pGEX4T3 expression vector. The 5′ oligonucleotide primer was constructed as follows. First, one base was inserted immediate upstream of the coding sequence (which does not include the ATG start codon, expression begins at the first ATG codon upstream of the glutathione-S-transferase gene that was originally constructed into the expression vector by the manufacturer). Second, an EcoRI restriction site was inserted 5′ to the beginning of the GSK3β Coding sequence. Third, three additional bases were placed 5′ to the EcoRI restriction site to facilitate the binding of the EcoRI restriction enzyme to the PCR amplified DNA product. The 3′ oligonudeotide primer was constructed as follows. An XhoI restriction site was inserted immediately downstream of the TGA stop codon. Subsequently, three bases were added 5′ to the XhoI restriction site. Sequence analysis of the corrected recombinant clone revealed no discordances with the 1263 bp original wild type sequence. Active GSK3β enzyme was expressed using the bacterial expression system. Expression of the fusion protein is under stringent control of the tac promoter which is inducible upon addition of a lactose analog, such as isopropyl β-D-thiogalactoside (IPTG). The host bacterial cell UT5600 was transformed by pGEX-4T3-GSK3β and grown in 2xYT medium supplemented with 100 μg/ml ampicillin. After induction of 150 μM IPTG at room temperature overnight, the UT5600[pGEX-4T3-GSK3β] cells are harvested and lysed using gentle sonication in the buffer (50 mM Tris-HCl, 1 mM EDTA, 500 mM NaCl, 1% Triton X-100, 1 mg/ml lysosyme, 1 mM benzamidine, 0.1 mM PMSF and 1 ug/ml soybean trypsin inhibitor). The recombinant GST-GSK3β protein was purified from the supematant using a GST-glutathione affinity system (Sigma) according to the manufacturer's instructions. Following batch binding of the fusion protein to glutathione-agarose beads, the matrix was transferred to a 2 inch diameter flex-column (Kontes Glass). The column was then washed with high-salt buffer (50 mM Tris-HCl, PH8.0, 1 mM EDTA and 500 mM NaCl) and low-salt buffer (50 mM Tris-HCl, PH8.0, 1 mM EDTA and 50 mM NaCl). Finally, the GST-GSK3β, fusion protein was eluted from the matrix using a glutathione buffer (50 mM Tris-HCl, PH7.5, 1 mM EDTA, 50 mM NaCl and 10 mM glutathione.)

B. GSK3β In Vitro Kinase Assay

Biochemical analysis was performed on recombinant human GSK3β fusion protein using the experimental protocol outlined in the section entitled “IN VITRO ACTIVITY PROFILE FOR KINASE”. Generally, the GST-GSK3β preparations were found to exhibit protein phosphotransferase activity in the order of 150 pmol/min/μg in the presence of 50 uM of [γ-³²P]-ATP and 69.3 uM of GSK substrate peptide (amino acid sequence: TRRAAVPPSPSLSRHSSPHQSEDEEE) in a 15 min reaction at ambient temperature.

### EXAMPLE 5

### In Vitro Activity Profile for Kinases

Compounds of formula (I) were tested in the following assay for their ability to inhibit the activity of the desired kinase, such as, PRKD2 and GSK3β. The desired in vitro potency of a particular inhibitor is such that the compound is useful as a therapeutic agent, i.e. in the nanomolar or micromolar range.

A. Assay Description

Test compounds were lyophilized and stored at −20° C. Stock solutions were made by weighing out the compounds and dissolving them in dimethyl sulfoxide (DMSO) to a standard concentration, usually 20 mM, and stored at −20° C. The compounds were diluted to a starting intermediate concentration of 250 μM in 1% DMSO, then serially diluted across a row of a 96 well plate using serial 2 fold dilution steps. Diluted 100% DMSO was used as a negative control.

5 μL of each compound dilution were robotically pipetted to Costar™ serocluster plates maintaining the same plate layout. All assay mixtures consisted of the following volumes:
5 μL diluted compound
10 μL target enzyme preparation
1 μL substrate
5 μL assay ATP

The assay mixtures were then incubated 15 minutes at ambient temperature.

From each assay mixture, 10 μL of assay mixture was spotted onto Millipore Multiscreen-PH™ opaque plates and washed twice for 10 minutes in 1% phosphoric acid. The plates were dried at 40° C. for 30 minutes, then the substrate phosphate complexes were quantitated by scintillation counting. These Millipore plates are in a 96-well format with immobilized P81 phosphocellulose membranes in the wells. Both the phosphorylated and non-phosphorylated form of the substrate bind to the membrane while ATP (unincorporated phosphate) is removed in the subsequent wash steps.

B. Calculation of IC₅₀

Inhibition of the targets by the test compounds is measured by scintillation counting of the incorporation of radioactive phosphate onto a specific substrate which is immobilized onto a filter paper at the end of the assay. To provide meaningful measurements of inhibition, the assays are performed both in the absence and presence of specific and known inhibitors, and the amount of incorporated radioactivity is compared to provide a baseline measurement.

The “baseline activity” is the amount of radioactivity incorporated in the absence of a target inhibitor. The amount of radioactivity incorporated in the presence of a target inhibitor is called the “sample activity”, and the % inhibition is expressed by the following formula:
% inhibition=100−(sample activity/baseline activity*100)

and is usually expressed in conjunction with the compound concentration. By using a range of target inhibitor concentrations, the IC₅₀ of an inhibitor is estimated (i.e. the concentration at which enzymatic activity is reduced by 50%). The IC₅₀ of various inhibitors against a particular target can be compared, where a lower IC₅₀ indicates a more potent inhibitor.

The IC₅₀ for 5-[5-(4-bromophenyl)-furan-2-ylmethylene]-thiazolidine-2,4-dithione against PRKD2 in vitro was 4.7. The IC₅₀ for 5-[5-(4-bromophenyl)furan-2-ylmethylene]-thiazolidine-2,4-dithione against GSK3β in vitro was 5.2.

### EXAMPLE 6

### Cell Migration in a Boyden Chamber

A range of cell lines are used in this assay, particularly the prostate cancer cell line PC3 and PTPN12 mouse embryonic fibroblasts (MEFs). The role of PTPN12 in migration was established based on the observations of PTPN12 negative MEFs. Cell adhesion and migration are dynamic biological activities involving the assembly and disassembly of a large number of extracellular and intracellular molecules, for example, actin, which are regulated in turn by protein phosphorylation. Hence locking the system in a phosphorylated (inhibition of phosphatases) or dephosphorylated (inhibition of kinases) state has a profound effect on the assembly/disassembly process and ultimately, migration. Migration is reduced in PTPN12 knock-out MEFs. By extension, a PTPN12 inhibitor should reduce cell migration in a Boyden chamber. Therefore, as a readout for PTPN12 activity, the following assay is designed to analyze cell migration in Boyden chambers. The Boyden assay is an experiment used to determine the capacity of a cell type to migrate on extracellular matrix. Unless otherwise indicated, all procedures are performed under sterile conditions in a flow laminar hood and all incubations at 37° C. are performed in the CO₂ incubator.

A. Reagents

1. Staining Solution.

Calcein AM (Molecular Probes, Cat# C-1430) stain is prepared at 0.5 ug/ml in Hanks buffered saline solution (GIBCO/BRL, Cat#14170-112).

2. Fibronectin Solution

A stock solution of fibronectin is prepared by dissolving 5 mg of fibronectin: (Sigma, Cat: F-2006) in 5 mL of sterile phosphate-buffered solution (PBS) by up and down agitation with a P1000 pipette. The working solution is prepared by mixing 100 ul of this stock solution with 10 mL of sterile PBS.

B. Assay (Tumour Cell Lines)

For tumour cell lines, stock cells (i.e. PC3 cells) are grown to 50-70% confluency in T175 flasks. Cells are trypsinized and a suspension prepared to a concentration of 2×10⁵/ml in media without serum. To the top chamber of each well of the HTS FluoroBlok™ 24-well insert system plates (Cat# 351158) is added 450 βl of cell suspension (or media for controls). Compounds for testing are prepared as 10×stocks in serum-free media from DMSO stocks, with a maximum final DMSO concentration of 0.25%. 50 μl of compound (or DMSO control) is then added to each top chamber, while 750 μl of media containing 10% fetal bovine serum is added to the bottom chamber as the chemoattractant. The plates are incubated for 20-24 hours at 37° C., 5% CO₂. Following incubation, the insert plate is transferred into a second 24-well companion plate containing 0.5 ml of 5 ug/ml calcein AM in HBSS and incubated for 1 hour at 37° C., 5% CO₂. Fluorescence of migrated cells is read in a Fluoroskan Ascent FL™ (or equivalent) with bottom reading at excitation/emission wavelength of 485/538 nm. Only those cells that have migrated through the pores of the FluoroBlok™ membrane will be read. For MEFs, the plates are coated on both sides of the membrane with 10 mg/mL fibronectin solution for 18 hours at 4° C. After incubation, the coating solution is removed by aspiration and the excess is washed twice with PBS. Cell seeding and detection are then performed as described for tumour cell lines.

C. Data Analysis

Data is expressed as fluorescence unit (FU) from the sum of middle 25 areas per 24-well or as percentage of migration inhibition by following formula: % of invasion inhibition=100−FU of compound treated cell invasion/FU of DMSO treated cell invasion times 100. Background is subtracted from all values, with background being represented by the media only controls.

TABLE 2

| | % Inhibition of |
|---|---|
| Compound | Migration at 25 μM |
| 5-[5-(4-Bromophenyl)-furan-2-ylmethylene]- | 65 |
| thiazolidine-2,4-dithione | |

### EXAMPLE 7

### The Status of P130^{CAS} Phosphorylation on Western Blots

Phosphotyrosine profiling of PTPN12-heterozygote and PTPN12-knockout mouse fibroblasts showed that a protein migrating at 130 kDa is constitutively hyperphosphorylated in the knockout cells (Côté, J. F., et al., Biochemistry (1998), Vol. 37, No. 38, pp.13128-13137). This protein was identified as being p130^{cas}, a protein found in focal adhesion complexes. It also appeared that the hyperphosphorylation of p130^{cas} in the PTPN12 knockout cells resulted in defective cell motility and focal adhesion turnover (Angers-Loustau et al., 1999).

This following assay measures p130^{cas} phosphorylation status as a readout of PTPN12 or other PTP activity such as PTP-1B. Briefly, the general tyrosine phosphorylation state of all cellular proteins is reduced by incubating the cells in suspension and then plating the cells onto fibronectin-coated plates, thereby stimulating tyrosine phosphorylation through the integrin pathway. Following cell lysis; p130^{cas} immunoprecipitation and Western blotting using 4G10 antiphosphotyrosine antibody are used to measure the tyrosine phosphorylation status of p130^{cas}. A low level of p130^{cas} tyrosine phosphorylation is indicative of a high PTPN12 activity. The assay is performed using PTPN12 knockout and heterozygote mouse fibroblasts.

A. Materials

1. PTPN12 +/− mouse fibroblasts (AC4 +/−) and PTPN12 −/− mouse fibroblasts (AC6 −/−) as kindly provided by Michel Tremblay and colleagues from the Cancer Centre at McGill University.

2. RIPA Buffer is made by mixing 50 mM Tris-HCl pH 7.2, 150 mM NaCl, 0.1% SDS (BioShop, Cat#: SDS 001), 0.5% sodium deoxycholate 10% solution (Sigma, Cat: D-6750), 1% NP-40 (BDH Laboratory Supplies, Cat: 56009 2L), 1 mM sodium vanadate (Fisher Scientific, Cat: S454-50) 200 mM solution, and “complete protease inhibitor mixture” (Roche Cat. 1836153).

3. SDS sample buffer is prepared by mixing 62.5 mM Tris-HCl pH 6.8, 20% glycerol (BioShop, Cat#: Gly 001), 2% SDS, 5% β-mercaptoethanol (Acros Organics, Cat#: 12547-2500), and 0.025% bromophenol blue (EM Science, OmniPur™).

B. Fibronectin Stimulation

6-Well plates (Fisher Scientific, Cat: 08-772-1B, Falcon No. 3530) are coated for 18 hours at 4° C. with a 10 mg/mL fibronectin solution (Sigma, Cat: F-2006, Lot: 109H7602) (density of 1 g/cm²). A volume of 950 μl of the fibronectin solution is added to each well. The plates are washed 2 times by adding 2 mL of PBS at ambient temperature to each well and by removing the PBS by aspiration. PBS 1% BSA solution (2 mL) is added to each well to block non-specific sites and the plates are incubated for 1 hour at 37° C. in CO₂ incubator. The blocking solution is removed by aspiration and the wells are washed before adding the cells to the wells.

C. Addition of Cells

Before adding the cells (AC4 +/− and AC6 −/−) to the prepared plates, They are washed and removed from 10 cm culture dishes by incubating them for 10 minutes at 37° C. in the CO₂ incubator with 1.5 mL of Trypsin/EDTA (0.05% Trypsin, 0.53 mM EDTA) (GibcoBRL, Cat: 25300-054) solution. Detached cells are suspended in 5 mL of PBS at ambient temperature, placed in 15 mL conical tubes and centrifuged at 600 g on a clinical centrifuge for 5 minutes. PBS is removed by aspiration, then the cells are counted using a hemacytometer and cell concentration is adjusted to 1×106 cells/mL in DMEM 0.5% BSA.

The cell suspension mixed with a test compound in an amount adequate to provide a range of 25 to 50 μM concentration is incubated for 30 minutes at 37° C. in the CO₂ incubator with mixing every ten minutes. An aliquot is retained as a control to determine the basal phosphorylation level before fibronectin-treatment. For fibronectin treatment, 3 mL of the cell suspension is added to the fibronectin matrix in order to obtain 60% confluence (3×10⁸ cells/well) before incubating for 45 minutes at 37° C. in CO₂ incubator. Each sample is performed in duplicate.

At the end of fibronectin stimulation or incubation in suspension, cells are washed with ice-cold PBS supplemented with 1 mM sodium orthovanadate. Cells are lysed directly on the plate by adding 0.5 mL of ice-cold RIPA buffer supplemented with protease inhibitors and 1 mM sodium vanadate. Plates are incubated at 4° C. with frequent agitation for 10 minutes, then disrupted by repeated aspiration with a P1000™ micropipette before transfer to 1.5 mL microcentrifuge tubes. Cellular debris is pelleted at 13,000 rpm (10000 g) for 10 minutes at 4° C. in a microcentrifuge, and supematants are drawn off into fresh 1.5 mL microcentrifuge tubes

Protein concentration in the cell lysates is assayed using Bio-Rad Protein concentration kit DC™ (Bio-Rad) according to manufacturer's instructions. Immunoprecipitation of p130^{cas} is performed with an amount of 250 mg protein adjusted in a final volume of 1 mL with RIPA buffer supplemented with 1 mM vanadate and inhibitors.

For the immunoprecipitation, 1 mg (4 mL) of anti-p130^{cas} mouse monoclonal (Transduction Laboratories, Cat: P27820) is added to each sample and the mixture is incubated for 2 hours at 4° C. on a rotating device. As an immunoprecipitation control, the same amount of cell lysate is incubated at this step with 1 mg (3 mL) of rabbit pre-immune serum. Then 20 mL of resuspended Protein G-Agarose™ beads (GibcoBRL, Cat: 15920-010) is added and the mixture is incubated with agitation for 1 hour at 4° C. on a rotating device. Immunoprecipitates are collected by centrifugation at 2000 g for 5 minutes at 4° C. Pellets are washed 3 times with 1 mL of ice-cold RIPA buffer (the supematant is removed by aspiration). After final wash, the beads are resuspended into 60 mL of SDS sample buffer.

D. SDS-PAGE and Western Blotting

30 μl of immunoprecipitate are separated on a 10% polyacrylamide gel for 1.5 hours at 125V (p130^{cas} is a 130 kDa protein)

Briefly, nitrocellulose membranes are blocked with TBS-Tween (TBST): 20 mM Tris-HCl, pH 7.2-7.4 (BioShop, Cat#: TRS 001)), 150 mM NaCl: (BioShop, Cat#: SOD 001) and 0.1% (v/v) Tween-20: (BioShop, Cat: TWN508) 1% BSA for 1 hour with agitation at ambient temperature. Antiphosphotyrosine monoclonal antibody clone 4G10 (Upstate Biotechnologies) is used at a 1/1000 dilution in TBST 1% BSA and incubated for 1 hour with agitation at ambient temperature. The anti-mouse-lgG-HRP (horseradish peroxidase) conjugate (Jackson Laboratories) is used at a 1/20000 dilution in TBST 1% BSA and incubated for 1 hour at ambient temperature.

E. Data Analysis

The data are analyzed as a function of p130^{cas} phosphorylation status.

Compounds of the invention tested demonstrate a higher level of phosphorylation in the PTPN12 −/− cells when compared to the PTPN12 +/− cells after fibronectin-treatment. Inhibition of PTPN12 in the +/− cells by a compound of the invention results in a higher phosphorylation state of p130^{cas} in the treated cells when compared to the non-treated cells.

The foregoing assay is also used, with the appropriate starting reagents and enzyme preparations, to test the ability of the compounds of the invention to inhibit PTPN 12 activity.

### EXAMPLE 8

### Cell Proliferation

This procedure (Jelinkova, R. B. et al., “Antiproliferative effect of a lectin- and anti-Thy-1.2 antibody-targeted HPMA copolymer-bound doxorubicin on primary and metastatic human colorectal carcinoma and on human colorectal carcinoma transfected with the mouse Thy-1.2 gene”, Bioconjug. Chem. (2000, September-October), Vol. 11, No. 5, pp. 664-73) is used to assess the effect compounds have on various cell lines with respect to proliferation. The rate of anchorage-independent growth of various tumor cells is quantified by measuring the amount of free isotopic thymidine that has been incorporated into the cells over a period of time. The effect of any compound to inhibit the proliferation of various tumor cells could be used as an indication of its ability to prevent disease progression in cancer.

Cultured tumour cells are harvested cells as per normal procedures: i.e. trypsinize, centrifuge and count cells. A volume of 90 μL is used to seed 5,000 cells/well in a 96 well plate. Cells are incubated for 24 hours at 37° C. under 5% CO₂. After incubation, cells should be 80-90% confluent.

³H-thymidine (Amersham) is diluted in cell culture media to a concentration of 100 μCi/mL. The test compound is diluted in the thymidine broth to 10× the final desired concentration.

Then 10 μL of diluted compound is added to the 90 μL of cells already present in the 96-well plates. Six replicates wells are done per treatment in columns 2 to 11. Plates were mixed by rocking.

A known cytotoxic compound such as staurosporine is used in relatively high concentrations as a positive control in column 1. Diluted DMSO is used as a negative control in column 12. The plate is incubated for exactly 24 hours at 37° C.

After incubation, plates are observed under the microscope for obvious cell death, abnormal cell shape, crystal formation of the compound, etc. Then 25 μL volume of cold 50% TCA is added slowly to the 100 μL volume already in each well, and incubated for 1-2 hours at 4° C. The plates are then washed 5× in tap water and allowed to dry completely (usually overnight) at ambient temperature. Finally, 100 μL of scintillation fluid is added to each well and the plates are counted in a Wallac 1450 Microbeta™ counter according to user manual instructions.

The amount of inhibition is determined by the following formula:
% inhibition=100−[(AVG treatment−AVG positive control)/100(AVG negative control−AVG positive control)]

TABLE 3

| | % Inhibition of | |
|---|---|---|
| | Proliferation at 50 μM | |
| Compound | H460 Cells | PC3 Cells |
| 5-[5-(4-Bromophenyl)-furan-2-ylmethylene]- | 89 | 15 |
| thiazolidine-2,4-dithione | | |

### EXAMPLE 9

### Cytotoxicity Assay

This procedure is used to assess the effects compounds have on various cell lines with respect to cell viability. Cell viability is quantified using calcein AM ((3′,6′-Di(O-acetyl)2′,7′-bis[N,N-bis-(carboxymethyl)aminomethyl)]-fluorescein, tetraacetoxymethyl ester) and measuring its conversion to a fluorescent product (calcein) with a fluorimeter.

The principle of this assay is based on the presence of ubiquitous intracellular esterase activity found in live cells. By enzymatic reaction of esterase, non-fluorescent cell-permeant calcein AM is converted to the intensely fluorescent calcein. The polyanionic dye calcein is retained within live cells, producing a green fluorescence in live cells. It is a faster, safer, and better-correlated indicator of cytotoxicity than alternative methods (e.g. 3H-Thymidine incorporation). calcein AM is susceptible to hydrolysis when exposed to moisture, Therefore, prepare aqueous working solutions containing calcein AM immediately prior to use, and used within about one day.

A kit available to do this assay is “LIVE/DEAD® Viability/Cytotoxicity Kit (L-3224)” by Molecular Probes.

Cells were collected from tissue culture flasks and trypsinized, centrifuged, resuspended and counted. Cells were seeded to obtain 80-90% confluence (for normal cells, 10,000 cells/well (8000 cells/well for HUVEC cells)). A cell concentration of 110,000 cells/mL (88,000 cells/well for HUVEC cells) is prepared as 90 μL volume is used per well.

Using an 8-channel multi-dispense pipettor, cells were seeded in the central rows of the plate (Nunclon™ 96 well flat-bottom plate), leaving the peripheral top and bottom rows with same volume of media only. The plates were incubated at 37° C., 5% CO₂ overnight for approximately 24 hours.

For test compounds, cell culture media (e.g., RPMI+10% FBS), 10×compound solution of final desired concentration from 20 mM stock compounds was prepared.

10 μl of this 10×compound solution is added to the 90 μL of cells already present in the 96 well plates and a known cytotoxic compound from previous testing is used as a positive control. The negative control is 100% DMSO diluted to the same factor as the compounds.

The plates are incubated at 37° C. for approximately 24 hours, and media is aspirated after plates are spun at 2400 rpm for 10 min at ambient temperature. 100 μL of 1×DPBS (without calcium chloride, without magnesium chloride (GibcoBRL, cat#14190-144)) is added to each well.

The calcein AM solution is prepared by added 50 μg of calcein AM crystal (m.w.=994.87 g/mol, Molecular Probes) and anhydrous DMSO (Sigma Aldrich) to make 1 mM stock and diluting stock to 2× the final desired concentration in 1×DPBS just before the assay. 100 μL of this 2× is added to the 100 μL of DPBS in the wells and the plates are incubated at ambient temperature for 30 minutes. Fluorescence data is read and recorded (Fluoroskan Ascent® FL fluorimeter (excitation˜485 nm, emission˜527 nm)).

The values for replicates (usually six) are averaged and % inhibition is calculated as follows:
% inhibition=100−[(AVG treatment−AVG positive control)/(AVG negative control−AVG positive control)*100]

TABLE 4

| | % Cytotoxicity On | | |
|---|---|---|---|
| | Normal Cells At 50 Mm | | |
| Compound | HS27 cells | Huvec cells | LL-86 cells |
| 5-[5-(4-Bromophenyl)-furan-2- | 24 | 47 | 11 |
| ylmethylene]-thiazolidine- | | | |
| 2,4-dithione | | | |

### EXAMPLE 10

### In Vivo Tumour Efficacy Study

To test the efficacy of test compounds on H460 subcutaneous xenograft alone and in combination with doxorubicin.

Athymic nude female mice are used for this experiment. A group of 60 mice are inoculated with five million H460 cells in 100 μL Matrigel™(VWR Canada) excipient. Tumours are measured three times a week with digital calipers and the tumour volumes calculated. When tumours have reached an average size of 100 mm³, about two weeks after tumour implantation. At that time any nongrowing ‘outliers’ are removed so that animals can be distributed into groupings that are equal and statistically the same tumour mass, i.e. divided into six groups with about 10 mice per group.

Treatments with test compounds continue for about 20 days, and will be oral (gavage), intravenous, subcutaneous, or intraperitoneal depending on the known solubility of the test compound. A dose of 25 mg/kg is typical for such testing, but the dose selected will reflect the potencty of the compound and the route of administration. Up to 200 mg/kg may be selected.

Positive controls may alternately be doxorubicin or cisplatin, or cyclophosphamide.

The study breakdown in tabular form:

| | | | | | 2^{nd} | Dose | | |
|---|---|---|---|---|---|---|---|---|
| Group | Treatment | Dose | Route | Schedule | Treatment | mg/kg | Route | Schedule |
| A | PTE | — | — | — | None | — | — | |
| B | Compound | 25 mg/kg | I.P. | Daily for 20 days | None | — | — | |
| C | Vehicle | — | I.P. | Daily for 20 days | Doxorubicin | 5 | IV | Every 4 days |
| D | Vehicle | — | I.P. | Daily for 20 days | Doxorubicin | 7 | IV | Every 4 days |
| E | Compound | 25 mg/kg | I.P. | Daily for 20 days | Doxorubicin | 5 | IV | Every 4 days |
| F | Compound | 25 mg/kg | I.P. | Daily for 20 days | Doxorubicin | 7 | IV | Every 4 days |

At study termination, the mice are anesthetized 3 hours after the last dose of test compound, and plasma and tissues are harvested and frozen. Tumours are divided into the desired number of aliquots and fast frozen for later analysis.

### EXAMPLE 11

### Cell Invasion in Matrigel™

This procedure is used to assess the compound effect on the tumor cell invasion through Matrigel™-coated Fluoroblok™ inserts. Invasion allows tumor cells to spread to sites other that the primary tumor. BD Bioscience's BioCoat FluoroBlok™ Invasion Systems™ combine the benefits of the BD BioCoat Matrigel™ Invasion Chambers with the fluorescence blocking membrane capabilities of the BD Falcon™ HTS FluoroBlok™ 24-Multiwell Insert System. The following assay uses this system to assess compound effects on the anti-tumor cell invasion through layer of Matrigel™ extracellular matrix.

The cell lines used are HT 1080 (ATCC, Cat# CCL-121), DU-145 (ATCC, Cat# HTB-81), PC3 (ATCC, Cat# CRL-1435) or B16F1 (ATCC, Cat# CRL-6323).

The invasion test system is removed from the package from −20° C. storage and allowed to warm to ambient temperature. PBS is added to the interior of the inserts and they are allowed to rehydrate for 2 hours at 37° C. Then the medium is removed and 450 μL cell suspensions of tumour cells (grown to 50-70% confluence, trypsinized, and resuspended in medium without serum at 1×10⁶/mL) is added to the top chamber. Test compounds are added to the top chamber at 10× the desired final concentration in 50 μL volumes. DMSO acts as control.

Then 750 μL of medium containing 50% fresh growth medium with 10% FBS and 50% NIH 3T3-conditioned medium is added to each of the bottom wells. The invasion system is then incubated for 24 to 48 hours at 37° C., in a 5% CO₂ atmosphere.

Following incubation, the insert plate is transferred into a second 24-well plate containing 0.5 mL of 5 μg/mL calcein AM (Molecular Probes) in Hanks buffered salt solution (HBSS), and plates are incubated for 1 hour at 37° C., 5% CO₂.

Fluorescence data indicating cell invasion is read in a Fluoroskan Ascent FL™ (LabSystems) with bottom reading at excitation/emission wavelength of 485/538 nm.

Data is expressed as fluorescence unit (FU) from the sum of middle 25 areas per 24-well or as percentage of invasion inhibition by following formula: % of invasion inhibition=100−FU of compound treated cell invasion/FU of DMSO treated cell invasion times 100.

The compounds inhibit invasion in this assay, and thus may be used to prevent metastasis in cancer and tissue remodeling.

### EXAMPLE 12

### Peritoneal Macrophage Stimulation and Analysis

A. Establishment of Inflammation Assay Panel.

Macrophages are important elements of innate immunity to infection and are among the first cell type in the immune response to be exposed to and activated by infectious agents. IFN-γ and LPS are potent activators of macrophages, priming them for a variety of biological effects. IFN-γ, initially secreted by NK and T cells in response to infection, converts macrophages from a resting to an activated state (inflammatory macrophages), priming them for antimicrobial activity manifested by increased killing of intracellular pathogens, and antigen processing and presentation to lymphocytes. The action of IFN-γ is synergized with the LPS second messenger, enhancing the stimulation of macrophages through the activation of NF-κB, that results in the transcriptional up-regulation of a number of genes involved in the cell-mediated immune response, including inducible iNOS (nitric oxide synthase). Activated macrophages are qualitatively different from quiescent macrophages. These differences are typically observed by an increased proliferation index, up-regulated expression of MHC-II, and production of various bioactive molecules. The latter biological effects are mediated by NO (nitric oxide) release and increased production of pro-inflammatory cytokines (IL-6, TNF-γ, IL-1). Primary macrophages derived from Balb/c mice and RAW 264.7 cells (Balb/c background) were used to establish in vitro inflammatory models with fast and reliable readouts.

B. Materials and Methods

1. Reagents.

The iNOS inhibitor NG-monomethyl-L-arginine (L-NMMA) and murine rlFN-γ are purchased from Calbiochem, (San Diego, Calif.). Protein-free, phenol/water-extracted LPS (from E. coli serotype 0111:B4 0127:B8), Zymosan A™, dexamethasone and hydrocortisone, sulfanilamide and N-(1-naphthyl)ethylenediamine, arare purchased from Sigma (St. Louis, Mo.). Human recombinant vascular endothelial growth factor (VEGF) is purchased from R&D Systems (Minneapolis, Minn.). Rabbit polyclonal antibody against active (phosphorylated) extracellular signal-regulated kinase (ERK), as well as HRP-conjugated donkey anti-rabbit IgG are obtained from Promega (Madison, Wis.). ELISA dual-set kit for detection of IL-6 is purchased from PharMingen (San Diego, Calif.). Anti-murine iNOS/NOS type II and cyclooxygenase-2 (COX-2) antibodies are obtained from Transduction Laboratories (Lexington, Ky.).

Female BALB/c inbred mice, 6-12 weeks of age, are purchased from Harlan Inc. (Indianapolis, Ind.) and housed under fluorescent light for 12 h per day. Mice are housed in cages, and maintained in compliance with the Canadian Council on Animal Care standards.

2. Isolation of Primary Mouse Macrophages.

Peritoneal exudate macrophages are isolated by peritoneal lavage with ice-cold sterile physiological saline 24 hours after intraperitoneal injection of BALB/c mice with 0.5 mL of sterile Zymosan A™ (1 mg/0.5 mL 0.9% saline). Cells are washed, resuspended in RPMI 1640 supplemented with 1 mM D-glucose, 1 mM sodium pyruvate, 100 units/mL penicillin, 100 μg/mL streptomycin, and 5% FBS.

3. Treatment of Primary Macrophages.

Primary macrophages (1.5×105 cells/well) are grown in 96-well plates (nitrite assay), or 6-well plates (2×106 cells/well) for measurement of iNOS and COX-2 expression. Following 3 hours incubation, at 37° C., 5% CO₂ (allowing macrophages to attach) cells are stimulated with LPS (5 μpg/mL) and IFN-γ (100 U/mL) in the absence or presence of various concentrations of test compounds (all treatments are replicated six times). Cells are incubated for an additional 24 hours, and cell free culture supematants from each well are collected for NO and cytokine determination. The remaining cells are stained with crystal violet or MTS to determine effect of the test compounds on cell survival.

4. NO Production.

Following stimulation, the production of NO is determined by assaying culture supernatants for NO₂, a stable reaction product of NO with molecular oxygen. Briefly, 100 μL of culture supematant is reacted with an equal volume of Griess reagent at ambient temperature for 10 minutes. The absorbance at 550 nm is determined. All measurements are performed six times. The concentration of NO₂ is calculated by comparison with a standard curve prepared using NaNO₂.

5. Western Blot Analysis.

After incubation with the indicated stimuli in the presence of inhibitors, cells (duplicate samples, 2×10⁶cell/6-wells plate) are washed in PBS and lysed on ice in 60 μL of lysis buffer. The protein content of each sample is determined using the Bradford protein assay kit (Bio-Rad, Richmond, Calif.). Absorbance is measured at 750 nm with a Beckman DU530 spectrophotometer (Palo Alto, Calif.). Proteins are mixed with 45×SDS sample buffer. Following separation of proteins by SDS-PAGE, using 8% bis-acrylamide in the separation gel, the proteins are transferred from the gels onto PVDF membranes using a MiniProtean™ III Cell (Bio-Rad), at 100 V for 1.5 hours. Equal amounts of protein (5 μg) are loaded onto SDS-PAGE gels and examined by Western blot analysis with anti-actin, anti-iNOS, anti-COX-2 murine monoclonal antibodies, according to the manufacturer's specifications (Transduction Laboratories). Primary antibodies, in 5% blocking buffer (5% NFM/TTBS), are incubated with blots 2 hours or ovemight at 4° C., followed by incubation with peroxidase-conjugated secondary antibody. Chemiluminescence substrates are used to reveal positive bands. The bands are exposed on X-ray films. The films are used to analyze the impact of inhibitors on expression of iNOS and COX-2 compared to various controls and “house-keeping” protein (Actin) concentration to control the protein loading and detect any non-specific effects on protein production. The Multi-Analyst™/PC system from Biorad is used to quantitate the bands of the expressed protein on the film. This version of Multi-Analyst is used with the Bio-Rad Gel Doc 1000™ imaging system. White light is chosen as the selected light source, thus the signal strength is measured in OD (optic density) units. The OD of each band is being subtracted to a global background area of the gel.

C. In Vitro Angiogenesis.

HUVEC cells cultured for 24 hours in M199 with 0.5% FCS are plated at 6×105 cells/well in 12-well plates pre-coated with 300 μL of Matrigel (10.7 mg/mL; Becton Dickinson) in M199 with 0.5% FCS in the presence of VEGF (1 ng/mL), and in the absence or presence of positive control (Z)-3-[2,4-dimethyl-5-(2-oxo-1,2-dihydroindol-3-ylidenemethyl)-1H-pyrrol-3-yl]propionic acid or various inhibitors. After 5 hours of incubation in a 5% CO₂-humidified atmosphere at 37° C., the three-dimensional organization of the cells is examined using an inverted photomicroscope. The cells are fixed with Crystal Violet (0.05% in 20% ethanol) and digitally photographed.

D. Enzyme Immunoassays for Mouse IL-6.

IL-6 levels are determined with PharMingen's OptEIA ELISA set developed using an anti-mouse IL-6 Ab pair and mouse rIL-6 standard (PharMingen). Maxisorp F16 multiwell strips (Nunc, Roskilde, Denmark) are coated with anti-mouse IL-6 capture Ab (at recommended concentration) in 0.1 M NaHCO₃, pH 9.5, 100 μL/well, overnight at 4° C. Plates are washed three times with 0.05% Tween 20 in PBS (PBST) and blocked for 1 hour at ambient temperature with 200 μL/well of 10% FCS in PBS (blocking and dilution buffer). Plates are washed three times with PBST and duplicate samples (100 μL/well) or standards (100 μL/well) in diluent buffer are incubated for 2 hours at ambient temperature. Plates are washed five times with PBST and incubated with biotinylated anti-mouse IL-6 and avidin-horseradish peroxidase conjugate (at concentrations recommended by the manufacturer) for 1 hour at ambient temperature. Plates are washed seven times with PBST and 100 μL of 3,3′5,5′tetramethylbenzidine substrate solution (TMB substrate reagent set, BD PharMingen) is added to each well. After 15-30 minute incubation at ambient temperature, color development is terminated by adding 50 μL of 2 N H₂SO₄ (Sigma). Absorbance is read at 450 nm with an EL 312e microplate reader or the like. The lower limit of detection for IL-6 is 15.6 pg/mL.

### EXAMPLE 13

### NIDDM Model

In vivo oral treatment with formula (I) or formula (Ia) compounds of the invention result in significant glucose lowering in several rodent models of diabetes. In db/db mice, oral administration of the compounds elicited significant correction of hyperglycemia. In a streptozotocin-induced diabetic mouse model, compounds potentiate the glucose-lowering effect of insulin.

In normal rats, compounds improve oral glucose tolerance with significant reduction in insulin release following glucose challenge. A structurally related inactive analog is not effective on insulin receptor activation or glucose lowering in db/db mice.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1-23. (canceled)

24. A pharmaceutical composition useful in treating cancer or inflammation in a human, wherein the pharmaceutical composition comprises a pharmaceutically acceptable carrier, diluent or excipient and a compound of formula (I): 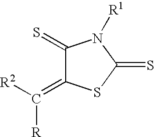
wherein:
R is heterocyclyl;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl; and
each R⁷ is independently hydrogen, alkyl or aralkyl;
as a single stereoisomer, a mixture of stereoisomers, or as a racemic mixture of stereoisomers; or as a solvate or polymorph; or as a pharmaceutically acceptable salt thereof;
provided, however, that when R¹ and R² are both hydrogen, R can not be unsubstituted thien-2-yl.

25. The pharmaceutical composition of claim 24 wherein the compound of formula (I) is a compound of formula (Ia): 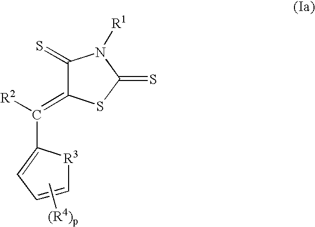
wherein:
p is 0 to 3;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
R³ is —O— or —S—;
each R⁴ is independently selected from the group consisting of alkyl, alkenyl, aryl, aralkyl, aralkenyl, cycloalkyl, cycloalkylalkyl, halo, haloalkyl, haloalkoxy, nitro, cyano, —R⁸—N═N—O—R⁷, —OR⁶, —C(O)OR⁶, —C(O)N(R⁶)₂, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —C(O)R⁶, —N(R⁶)C(O)N(R⁶)₂, —OC(O)N(R⁶)₂, or —N(R⁶)S(O)ₜR⁶ (where t is 0 to 2), heterocyclyl and heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl;
each R⁷ is independently hydrogen, alkyl or aralkyl; and
R⁸ is a direct bond or an optionally substituted straight or branched alkylene or alkenylene chain.

26. The pharmaceutical composition of claim 25 wherein the compound of formula (I) is a compound of formula (Ia) wherein:
p is 1;
R¹ is hydrogen, alkyl, or aralkyl;
R² is hydrogen or alkyl;
R³ is —O— or —S—; and
R⁴ is halo, haloalkyl, or haloalkoxy.

27. A compound of formula (I): 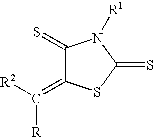
wherein:
R is heterocyclyl;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl; and
each R⁷ is independently hydrogen, alkyl or aralkyl;
as a single stereoisomer, a mixture of stereoisomers, or as a racemic mixture of stereoisomers; or as a solvate or polymorph; or as a pharmaceutically acceptable salt thereof;
provided, however, that when R¹ and R² are both hydrogen, R can not be unsubstituted thien-2-yl; and
provided, however, that when R¹ and R² are both hydrogen; R can not be unsubstituted furan-2-yl; 3-nitrofuran-2-yl, 4-nitrofuran-2-yl or 4-bromofuran-2-yl.

28. The compound of claim 27 of the formula (Ia): 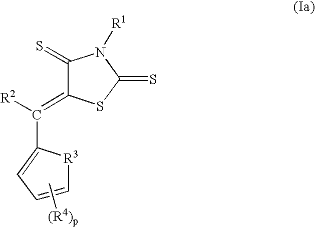
wherein:
p is 0 to 3;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
R³ is —O— or —S—;
each R⁴ is independently selected from the group consisting of alkyl, alkenyl, aryl, aralkyl, aralkenyl, cycloalkyl, cycloalkylalkyl, halo, haloalkyl, haloalkoxy, nitro, cyano, —R⁸—N═N—O—R⁷, —OR⁶, —C(O)OR⁶, —C(O)N(R⁶)₂, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —C(O)R⁶, —N(R⁶)C(O)N(R⁶)₂, —OC(O)N(R⁶)₂, or —N(R⁶)S(O)ₜR⁶ (where t is 0 to 2), heterocyclyl and heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl;
each R⁷ is independently hydrogen, alkyl or aralkyl; and
R⁸ is a direct bond or an optionally substituted straight or branched alkylene or alkenylene chain.

29. A method of treating cancer in a mammal, which method comprises administering to the mammal in need thereof a therapeutically effective amount of a compound of formula (I): 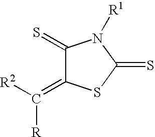
wherein:
R is heterocyclyl;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl; and
each R⁷ is independently hydrogen, alkyl or aralkyl;
as a single stereoisomer, a mixture of stereoisomers, or as a racemic mixture of stereoisomers; or as a solvate or polymorph; or as a pharmaceutically acceptable salt thereof.

30. The method of claim 29 wherein the compound of formula (I) is a compound of formula (Ia): 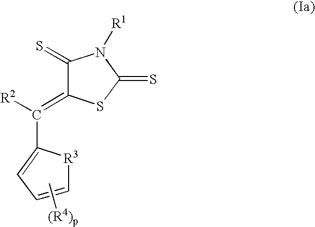
wherein:
p is 0 to 3;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
R³ is —O— or —S—;
each R⁴ is independently selected from the group consisting of alkyl, alkenyl, aryl, aralkyl, aralkenyl, cycloalkyl, cycloalkylalkyl, halo, haloalkyl, haloalkoxy, nitro, cyano, —R⁸—N═N—O—R⁷, —OR⁶, —C(O)OR⁶, —C(O)N(R⁶)₂, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —C(O)R⁶, —N(R⁶)C(O)N(R⁶)₂, —OC(O)N(R⁶)₂, or —N(R⁶)S(O)ₜR⁶ (where t is 0 to 2), heterocyclyl and heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl;
each R⁷ is independently hydrogen, alkyl or aralkyl; and
R⁸ is a direct bond or an optionally substituted straight or branched alkylene or alkenylene chain.

31. The method of claim 30 wherein the mammal is a human.

32. The method of claim 31 wherein the cancer is associated with hyperproliferation or tissue remodelling or repair.

33. The method of claim 32 wherein the cancer is associated with the activity of an enzyme selected from the group consisting of PTPN12, PTPN2, PRKD2, and GSK3β.

34. The method of claims 29-33 wherein the compound of formula (I) is a compound of formula (Ia) wherein:
p is 1;
R¹ is hydrogen, alkyl, or aralkyl;
R² is hydrogen or alkyl;
R³ is —O— or —S—; and
R⁴ is halo, haloalkyl, or haloalkoxy.

35. A method of treating inflammation in a mammal, which method comprises administering to the mammal in need thereof a therapeutically effective amount of a compound of formula (I): 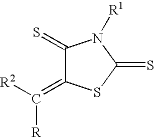
wherein:
R is heterocyclyi;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
each R⁵ is independently an optionally substituted straight or branched alkylene or alkenylene chain;
each R⁵ is independently hydrogen, alkyl, alkenyl, cycloalkyl, cycloalkylalkyl, aralkyl or aryl; and
each R⁷ is independently hydrogen, alkyl or aralkyl;
as a single stereoisomer, a mixture of stereoisomers, or as a racemic mixture of stereoisomers; or as a solvate or polymorph; or as a pharmaceutically acceptable salt thereof.

36. The method of claim 35 wherein the compound of formula (I) is a compound of formula (Ia): 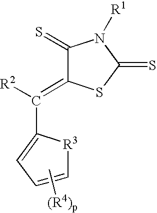
wherein:
p is 0 to 3;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
R³ is —O— or —S—;
each R⁴ is independently selected from the group consisting of alkyl, alkenyl, aryl, aralkyl, aralkenyl, cycloalkyl, cycloalkylalkyl, halo, haloalkyl, haloalkoxy, nitro, cyano, —R⁸—N═N—O—R⁷, —OR⁶, —C(O)OR⁶, —C(O)N(R⁶)₂, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —C(O)R⁶, —N(R⁶)C(O)N(R⁶)₂, —OC(O)N(R⁶)₂, or —N(R⁶)S(O)ₜR⁶ (where t is 0 to 2), heterocyclyl and heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl;
each R⁷ is independently hydrogen, alkyl or aralkyl; and
R⁸ is a direct bond or an optionally substituted straight or branched alkylene or alkenylene chain.

37. The method of claim 36 wherein the mammal is a human.

38. The method of claim 37 wherein the inflammation is associated with hyperproliferation or tissue remodelling or repair.

39. The method of claim 38 wherein the inflammation is associated with the activity of an enzyme selected from the group consisting of PTPN12, PTPN2, PRKD2, and GSK3β.

40. The method of claims 36-39 wherein the compound of formula (I) is a compound of formula (Ia) wherein:
p is 1;
R¹ is hydrogen, alkyl, or aralkyl;
R² is hydrogen or alkyl;
R³ is —O— or —S—; and
R⁴ is halo, haloalkyl, or haloalkoxy.

41. A method of treating a mammal having a disorder or condition associated with hyperproliferation and tissue remodelling or repair, wherein said method comprises administering to the mammal having the disorder or condition a therapeutically effective amount of a compound of formula (I): 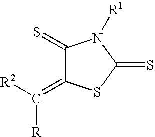
wherein:
R is heterocyclyl;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl; and
each R⁷ is independently hydrogen, alkyl or aralkyl;
as a single stereoisomer, a mixture of stereoisomers, or as a racemic mixture of stereoisomers; or as a solvate or polymorph; or as a pharmaceutically acceptable salt thereof.

42. The method of claim 41 wherein the compound of formula (I) is a compound of formula (Ia): 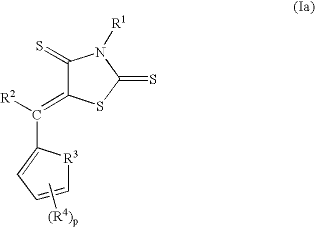
wherein:
p is 0 to 3;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
R³ is —O— or —S—;
each R⁴ is independently selected from the group consisting of alkyl, alkenyl, aryl, aralkyl, aralkenyl, cycloalkyl, cycloalkylalkyl, halo, haloalkyl, haloalkoxy, nitro, cyano, —R⁸—N═N—O—R⁷, —OR⁶, —C(O)OR⁶, —C(O)N(R⁶)₂, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —C(O)R⁶, —N(R⁶)C(O)N(R⁶)₂, —OC(O)N(R⁶)₂, or —N(R⁶)S(O)ₜR⁶ (where t is 0 to 2), heterocyclyl and heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl;
each R⁷ is independently hydrogen, alkyl or aralkyl; and
R⁸ is a direct bond or an optionally substituted straight or branched alkylene or alkenylene chain.

43. A method of treating a mammalian cell with a compound of formula (I): 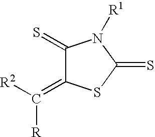
wherein:
R is heterocyclyl;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl; and
each R⁷ is independently hydrogen, alkyl or aralkyl;
as a single stereoisomer, a mixture of stereoisomers, or as a racemic mixture of stereoisomers; or as a solvate or polymorph; or as a pharmaceutically acceptable salt thereof;
wherein the method comprises administering the compound of formula (I) to a mammalian cell and the compound of formula (I) is capable of inhibiting the activity of PTPN12, PTPN2, PRKD2, and/or GSK3β within the mammalian cell.

44. The method of claim 43 wherein the compound of formula (I) is a compound of formula (Ia): 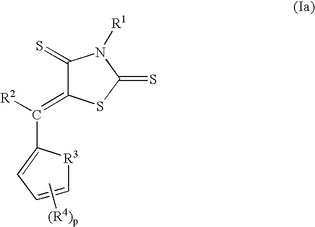
wherein:
p is 0 to 3;
R¹ is hydrogen, alkyl, alkenyl, aryl, aralkyl, aralkenyl, cycloalkyl, cycloalkylalkyl, haloalkyl, haloalkenyl, heterocyclylalkyl, —R⁵—O—R⁶, —R⁵—N(R⁶)₂, —R⁵—C(O)OR⁶, —R⁵—C(O)N(R⁶)₂, —R⁵—N═N—OR⁷, or —R⁵—N(R⁶)C(O)OR⁷;
R² is hydrogen, alkyl, aralkyl, aryl, haloalkyl, cycloalkyl, cycloalkylalkyl, heterocyclyl or heterocyclylalkyl;
R³ is —O— or —S—;
each R⁴ is independently selected from the group consisting of alkyl, alkenyl, aryl, aralkyl, aralkenyl, cycloalkyl, cycloalkylalkyl, halo, haloalkyl, haloalkoxy, nitro, cyano, —R⁸—N═N—O—R⁷, —OR⁶, —C(O)OR⁶, —C(O)N(R⁶)₂, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —C(O)R⁶, —N(R⁶)C(O)N(R⁶)₂, —OC(O)N(R⁶)₂, or —N(R⁶)S(O)ₜR⁶ (where t is 0 to 2), heterocyclyl and heterocyclylalkyl;
each R⁵ is independently an optionally substituted straight or branched alkylene or alkenylene chain;
each R⁶ is independently hydrogen, alkyl, alkenyl, cycloalkyl, cycloalkylalkyl, aralkyl or aryl;
each R⁷ is independently hydrogen, alkyl or aralkyl; and
R⁸ is a direct bond or an optionally substituted straight or branched alkylene or alkenylene chain.

45. The method of claim 41 wherein the mammalian cell is treated in vitro.

46. The method of claim 41 wherein the mammalian cell is treated in vivo.

47. The method of claim 41 wherein the inhibition of activity results in a reduction of cell adhesion.

48. The method of claim 41 wherein the inhibition of activity results in a reduction of cell division.

49. The method of claim 41, wherein the inhibition of activity results in a reduction of cell migration.

50. The method of claim 41, wherein the inhibition of activity results in control of tumor growth.

51. The method of claim 41 wherein the inhibition of activity results in control of lymphocyte activation.

52-56. (canceled)


SEQUENCE LISTING (4 sequences)

SEQ ID NO: 1 - protein, 9 aa, Unknown
  Mammalian tyrosine phosphatase conserved catalytic domain signature sequence
XHCXXGXXX

SEQ ID NO: 2 - protein, 13 aa, Artificial Sequence
  Synthesized in Laboratory
TSTEPQYQPGENL

SEQ ID NO: 3 - protein, 13 aa, Artificial Sequence
  Synthesized CREBtide(TM) substrate peptide
KRREILSRRPSYR

SEQ ID NO: 4 - protein, 26 aa, Artificial Sequence
  Synthesized GSK substrate peptide
TRRAAVPPSPSLSRHSSPHQSEDEEE